(19) United States
(12) Patent Application Publication
(10) Pub. No.: US 2020/0171110 A1
(43) Pub. Date: Jun. 4, 2020

(54) HSV VECTOR WITH REDUCED NEUROTOXICITY

(71) Applicant: Virogin Biotech Canada Ltd, Vancouver (CA)

(72) Inventors: William Jia, Burnaby (CA); Dmitry V. Chouljenko, Vancouver (CA); I-Fang Lee, Burnaby (CA); Yanal M. Murad, Vancouver (CA); Xiaohu Liu, Vancouver (CA); Guoyu Liu, Richmond (CA); Xuexian Bu, Surrey (CA); Zahid Delwar, Richmond (CA)

(21) Appl. No.: 16/699,248

(22) Filed: Nov. 29, 2019

Related U.S. Application Data
(60) Provisional application No. 62/773,119, filed on Nov. 29, 2018.

(51) Int. Cl. A61K 35/763; A61P 35/00; C12N 15/86; C07K 14/54; C12N 15/113; C07K 14/715

(52) U.S. Cl. CPC A61K 35/763; A61P 35/00; C12N 15/86; C07K 14/7155; C07K 14/5443; C12N 15/113; C07K 14/5434

(57) ABSTRACT

Recombinant herpes simplex viruses are provided having a modified oncolytic herpes virus genome, wherein the modified herpes virus genome has at least one miRNA target sequence operably linked to a first or to a first and a second copy of an ICP34.5 gene. Also provided are pharmaceutical compositions having such recombinant herpes simplex viruses, as well as methods of using such compositions in the treatment of subjects having cancer.

### CROSS-REFERENCE TO RELATED APPLICATION

This patent application claims the benefit under 35 U.S.C. § 119(e) of U.S. Provisional Patent Application No. 62/773,119 filed Nov. 29, 2018, which application is incorporated herein by reference in its entirety for all purposes.

### FIELD OF THE INVENTION

The present invention relates generally to HSV vectors having reduced neurotoxicity

### BACKGROUND

Oncolytic virotherapy has been recognized as a promising new therapeutic approach for cancer treatment because oncolytic viruses cause strong tumor oncolysis and induce a systemic tumor-specific immunity while causing significantly fewer side effects than chemotherapy or radiation treatments.

Among the various OVs, herpes simplex virus type 1 (“HSV-1”) based OVs are the farthest advanced, e.g., a herpes virus-based OV (T-Vec) has been approved by the U.S. FDA for the treatment of melanoma. Representative examples of HSV vectors include those described in U.S. Pat. Nos. 7,223,593, 7,537,924, 7,063,835, 7,063,851, 7,118,755, 8,277,818, and 8,680,068.

One difficulty with oncolytic herpes virus vectors is the neurotropic nature of HSV. Neuroinvasiveness is primarily mediated by the viral protein ICP34.5, leading to the common strategy of deleting ICP34.5 from vectors used in oncolytic virotherapy. However, complete deletion of ICP34.5 reduces the ability of the virus to replicate in a wide range of tissues by approximately 10-fold. The present invention overcomes certain difficulties associated with current HSV vectors, and further provides other, related advantages.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

Briefly stated, the application relates to recombinant herpes simplex viruses (also referred to as “oHSV vectors”) comprising at least one ICP34.5 gene having at least two miRNA target sequences in the 3′ untranslated region of ICP34.5. In certain embodiments, the at least two miRNA target sequences are targets for the same miRNA. In other embodiments, the at least two miRNA target sequences are targets for an miRNA selected from the group consisting of mIR-122, miR-124, miR-124*, miR-127, miR-128, miR-129, miR-129*, miR-132, mIR-133a, mIR133b, miR-135b, miR-136, miR-136*, miR-137, miR-139-5p, miR-143, mIR-145, miR-154, miR-184, miR-188, miR-204, mIR216a, miR-299, miR-300-3p, miR-300-5p, miR-323, miR-329, miR-337, miR-335, miR-341, miR-369-3p, miR-369-5p, miR-376a, miR-376a*, miR-376b-3p, miR-376b-5p, miR-376c, miR-377, miR-379, miR-379*, miR-382, miR-382*, miR-409-5p, miR-410, miR-411, miR-431, miR-433, miR-434, miR-451, miR-466b, miR-485, miR-495, miR-539, miR-541, miR-543*, miR-551b, miR-758, and miR-873. By convention, the strand that is more frequently found to be the final product is referred to as miRNA and the rarer partner as miRNA*.

In other embodiments, the recombinant herpes simplex virus further comprises a modified ICP27 or ICP4 gene, wherein the modification is a replacement of the 5′UTR, the promoter-regulatory region, or both the 5′UTR and the promoter-regulatory region. In some embodiments, the 5′UTR is derived from the FGF gene.

In certain embodiments, the recombinant herpes simplex virus further comprises a gene sequence encoding at least one immune stimulating factor, a checkpoint blocking peptide or both.

The disclosure also provides methods of treating cancer, comprising administering the recombinant herpes simplex virus comprising at least one ICP34.5 gene having at least two miRNA target sequences in the 3′ untranslated region of ICP34.5.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Other features, objects and advantages will be apparent from the description, the drawings, and the claims. In addition, the disclosures of all patents and patent applications referenced herein are incorporated by reference in their entirety.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 is a schematic of an exemplary HSV vector with three different miRNA targets in the 3′ untranslated region of ICP34.5.

FIG. 2 is a schematic of an exemplary HSV vector with a modified γ34.5 gene and a modified ICP4 or ICP27 gene.

FIG. 3 is a graph showing expression levels of ICP27, ICP4, and ICP47 in brains of normal mice and mice carrying a human brain tumor (U87).

FIG. 4 is a Western blot showing expression of ICP34.5 and β-actin in neuronal and tumor cells (LNCaP and A549).

FIG. 5 is a schematic of a transcriptional and translational dual-regulated virus.

FIG. 6 depicts various regulatory elements that may be used in the platform virus.

FIG. 7 are photographs of murine brain section following intracranial injection of either CXCR4-TF-Fc-h1215 virus or CXCR4-TF-Fc-h1215-miR virus. Brain sections were stained with rabbit polyclonal anti-HSV primary antibody and a fluorescent rat anti-rabbit secondary antibody.

FIGS. 8A, 8B, and 8C are graphs of cell survival following viral infection at various MOI. FIG. 8A shows cell survival for lung tumor cells A549 and normal lung cells BEAS-2b. FIG. 8B shows cell survival for lung tumor cells A549 and normal lung cells HPL1D. FIG. 8C shows cells survival for lung tumor cells A549, PC9, H460, H23S, H1975.

FIG. 9 is a graph showing replication of VG182LF virus in A549 lung tumor cells and BEAS-2b normal lung cells.

FIG. 10 is a bar chart showing increase (fold-increase) of IL-12 in A549 lung tumor cells and LNCaP prostate tumor cells following infection with hVG161 or hVG182LF.

FIGS. 11A, 11B, and 11C depict replication of VG182LF virus in various lung tumor cells. FIG. 11A: H1975 cells. FIG. 11B: H460 cells. FIG. 11C: PC9 cells.

FIG. 12 is a graph showing tumor size in nude mice bearing H1975 tumors at 1-week following treatment with either vehicle or VG182LF virus.

FIGS. 13A-13Z, 13AA-13ZZ, and 13AAA-13SSS are a selected list of microRNAs in tumors, which can be found on PubMed at https://www.ncbi.nlm.nih.gov/pubmed and on the microRNA database (“mIRBASE”) at http://www.mirbase.org/, all of which are incorporated by reference in their entirety.

FIGS. 14A, 14B, and 14C are graphs showing transfection efficiency of miR-143 at 6 hours post-infection, viral gene expression at 6 hours post-infection, and virus replication at 24 hours post-infection, respectively, in 293FT cells.

FIG. 15 are photographs showing HSV-1 immunostaining of murine brain and spinal cord sections. Mice were injected subcutaneously with either a control vehicle, wild-type HSV-1, a HSV-1 variant with deleted ICP34.5 (VG161) or a variant which encodes binding sites for miR-143 and miR-124 in the 3′ UTR of ICP34.5 along with a fusogenic mutation in the carboxyl terminus of gB (gB-876t) (VG301).

FIG. 16 is a graph showing the survival curve of mice injected subcutaneously with either wild-type HSV-1, a HSV-1 variant with deleted ICP34.5 (VG161) or a variant which encodes binding sites for miR-143 and miR-124 in the 3′ UTR of ICP34.5 along with a fusogenic mutation in the carboxyl terminus of gB (gB-876t) (VG301).

FIG. 17 are photographs showing results of a fusion assay in which cells were fixed and Giemsa stained to visualize viral plaques and syncytia resulting from virus-induced cell fusion. The cells were infected with recombinant oncolytic HSV-1 with (+gB-876t) or without (−gB-876t) a fusogenic mutation in the carboxyl terminus of gB.

### DETAILED DESCRIPTION OF THE INVENTION

The present invention may be understood more readily by reference to the following detailed description of preferred embodiments of the invention and the Examples included herein.

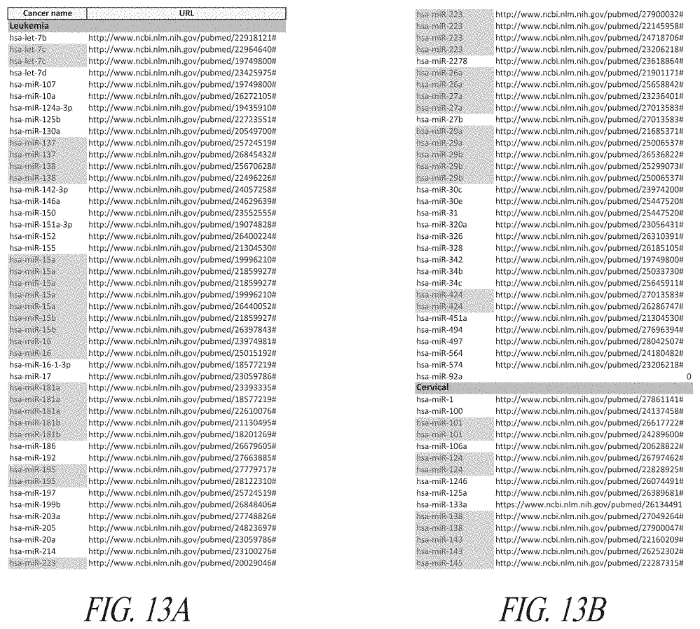

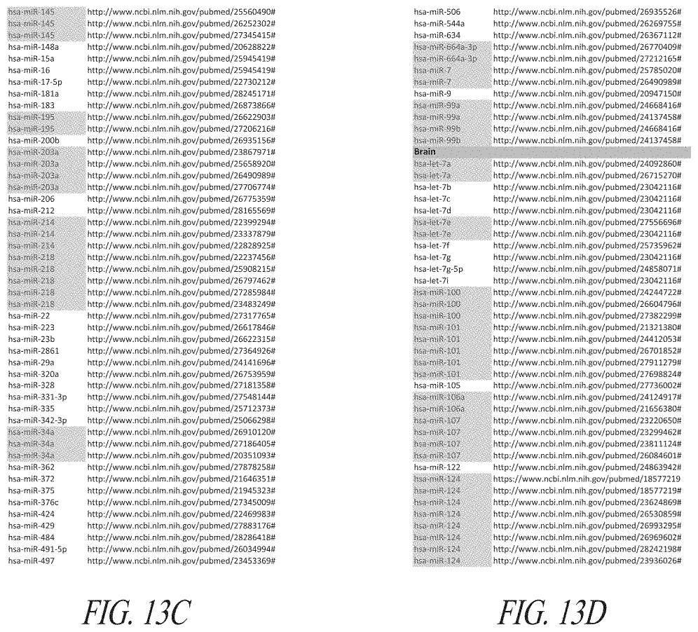

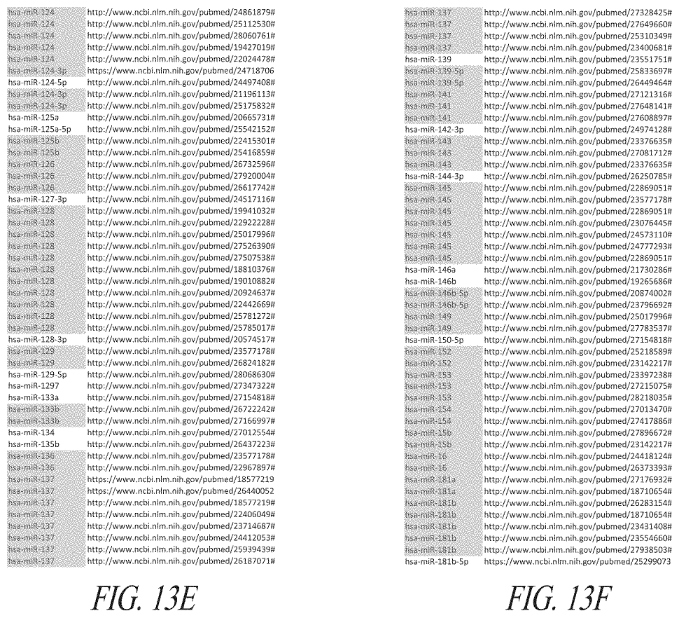

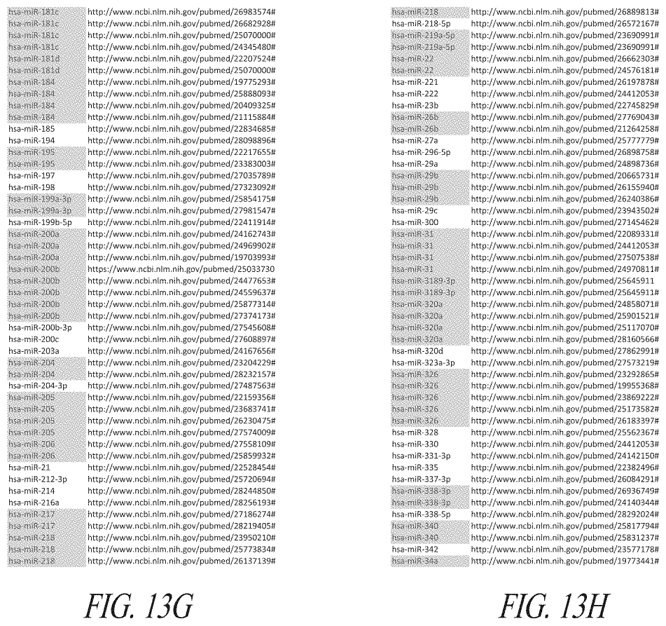

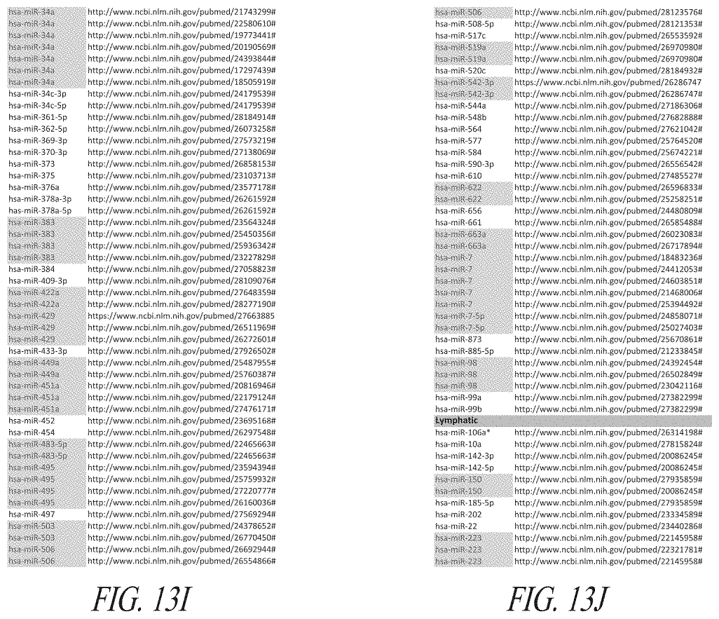

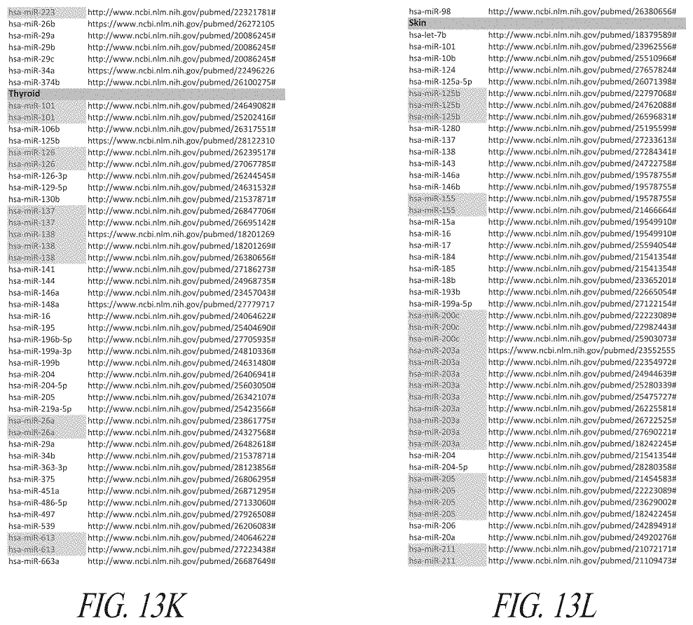

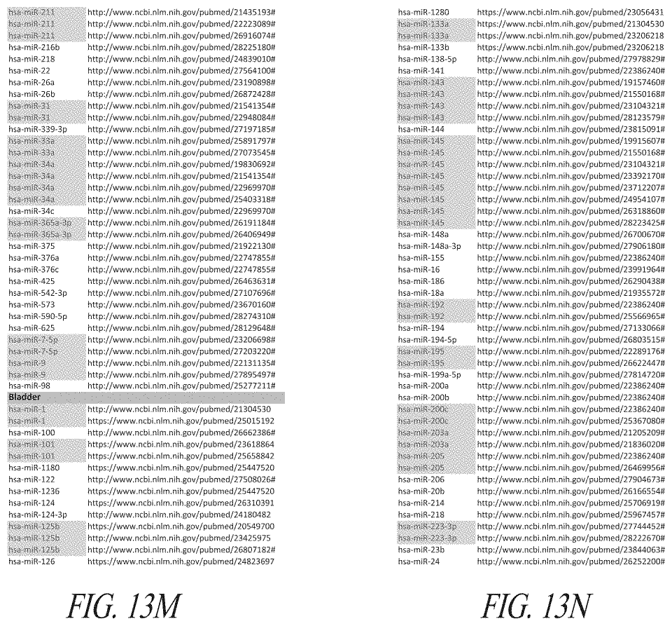

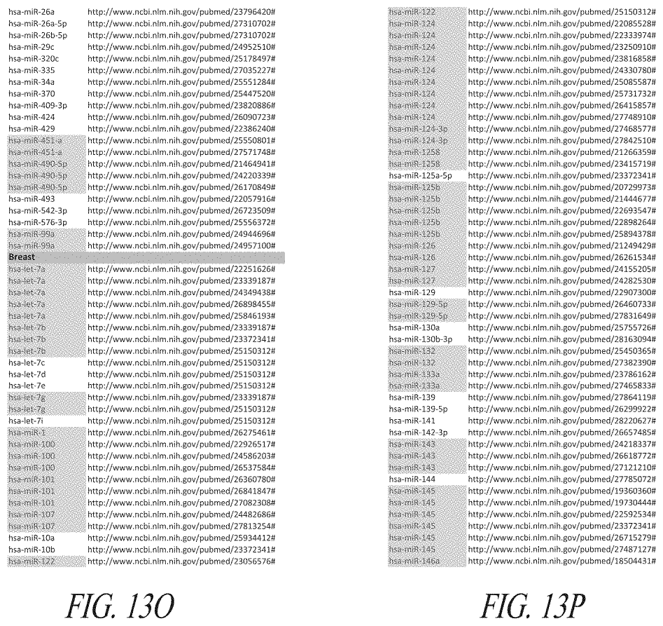

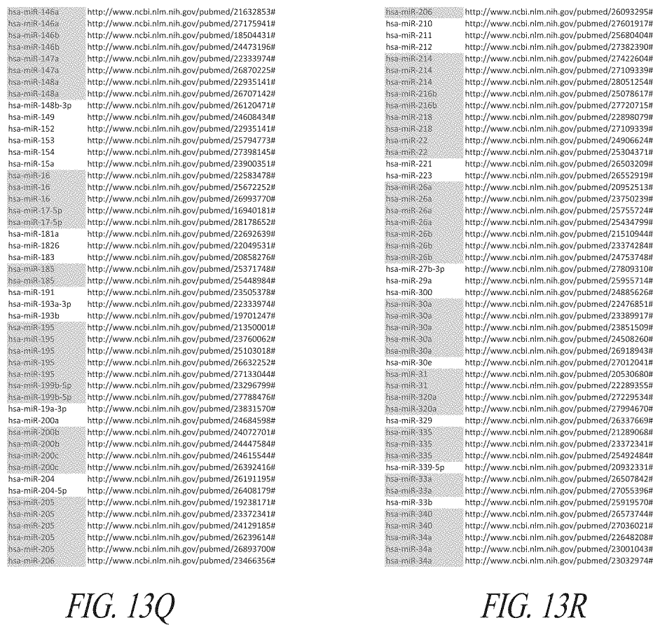

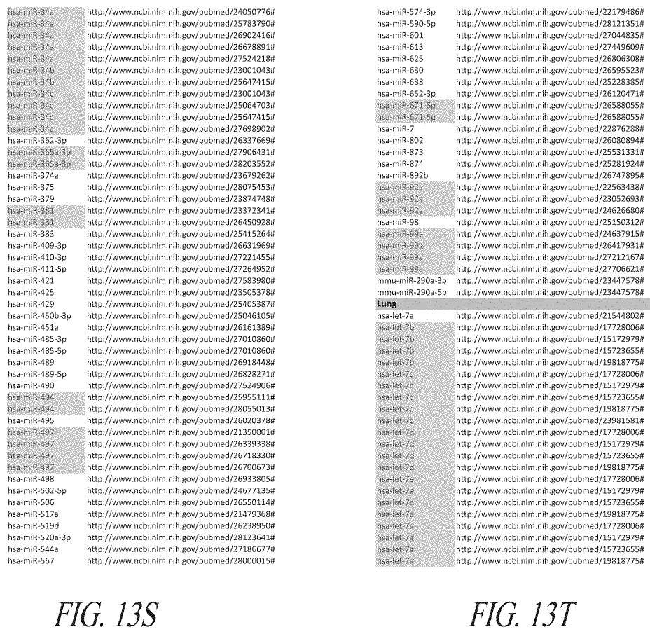

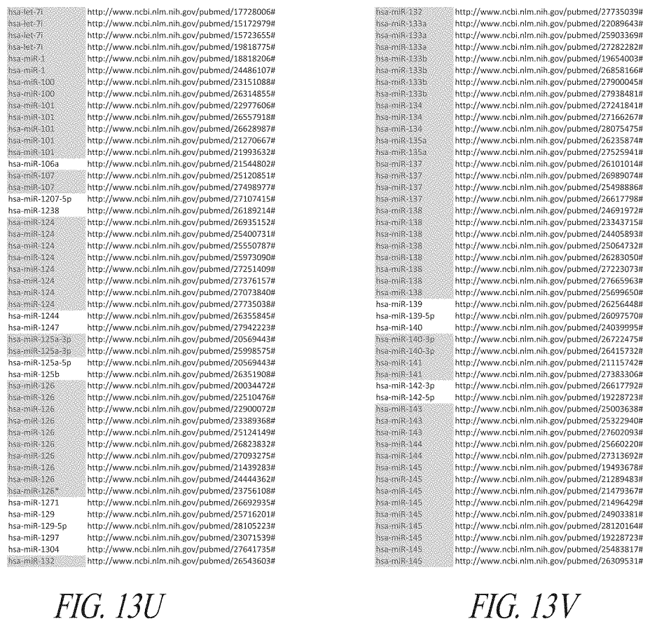

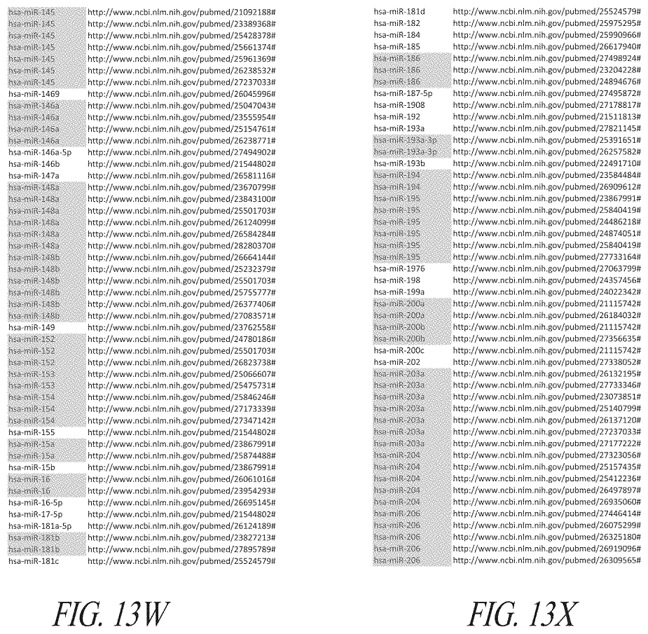

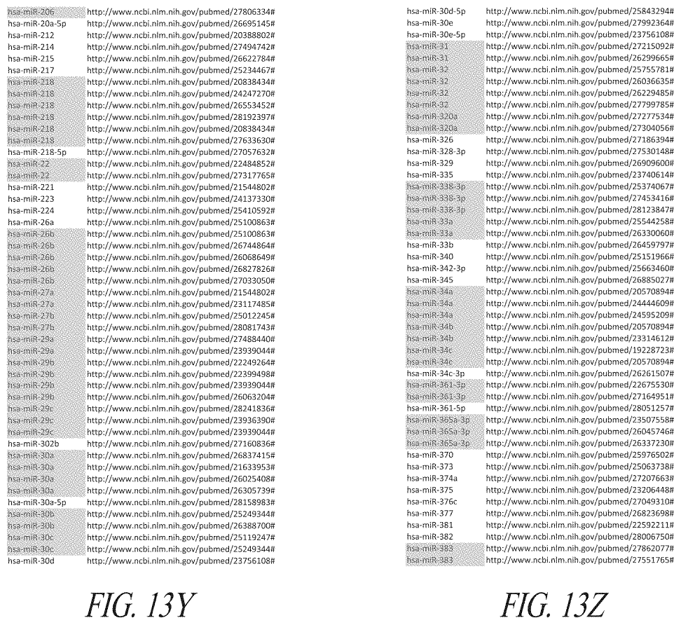

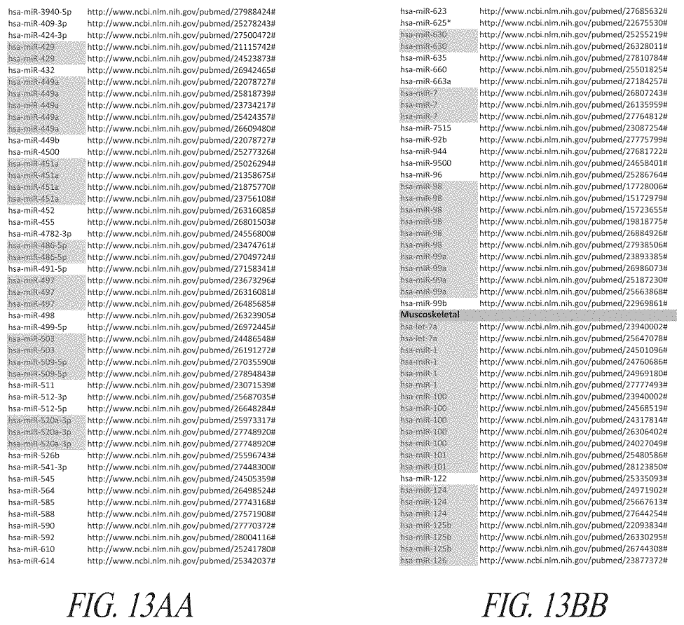

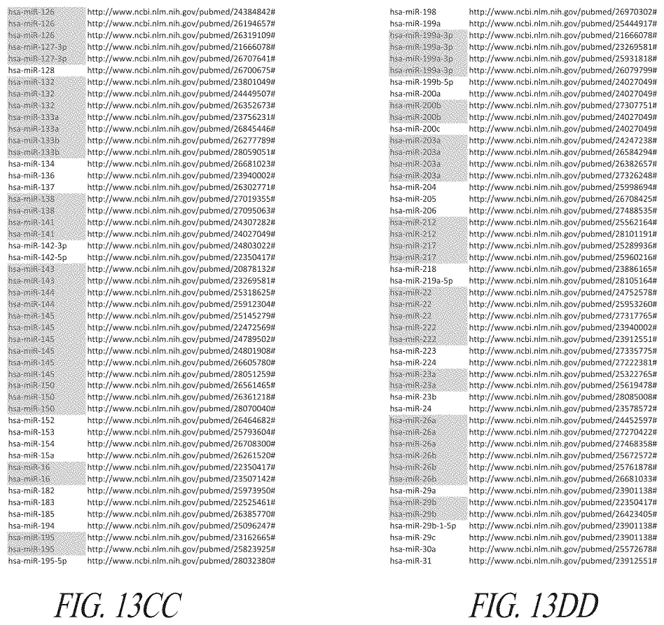

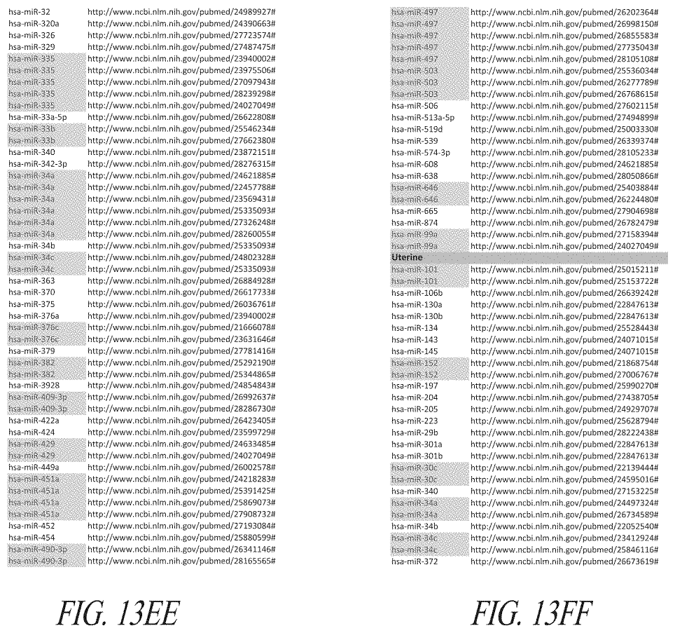

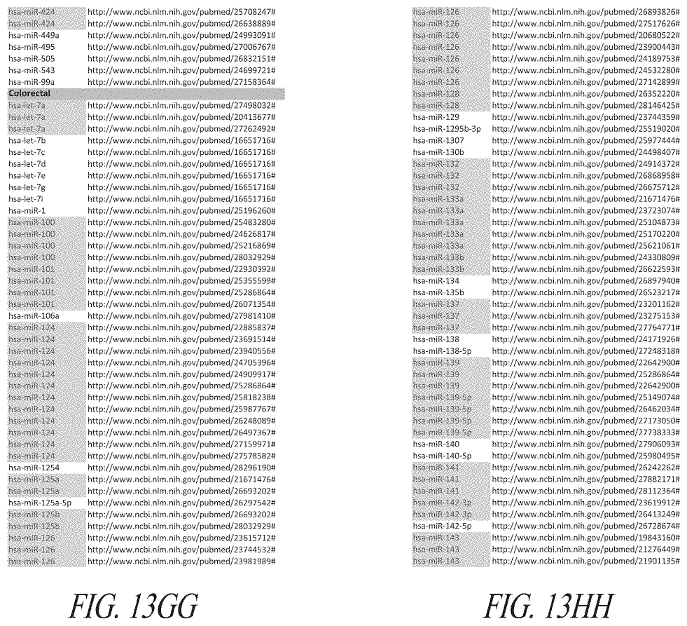

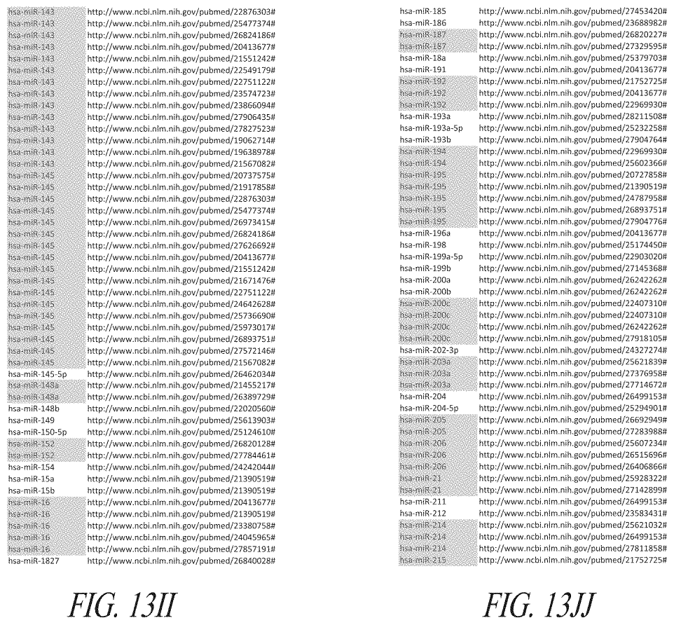

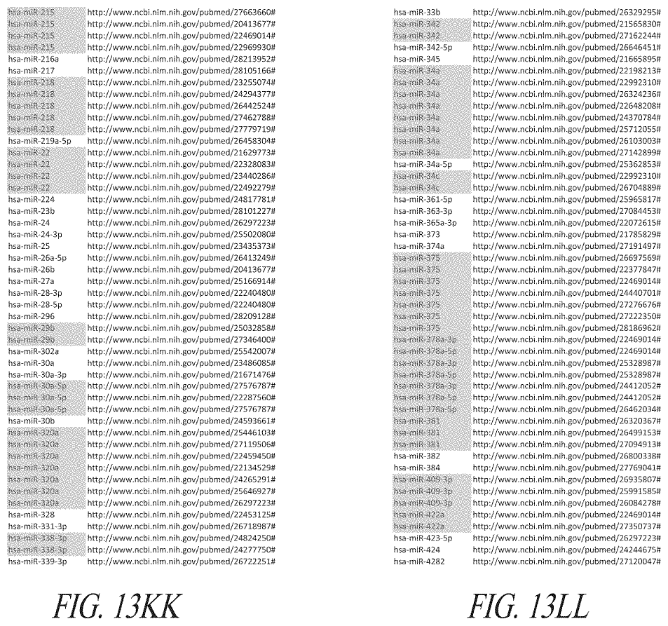

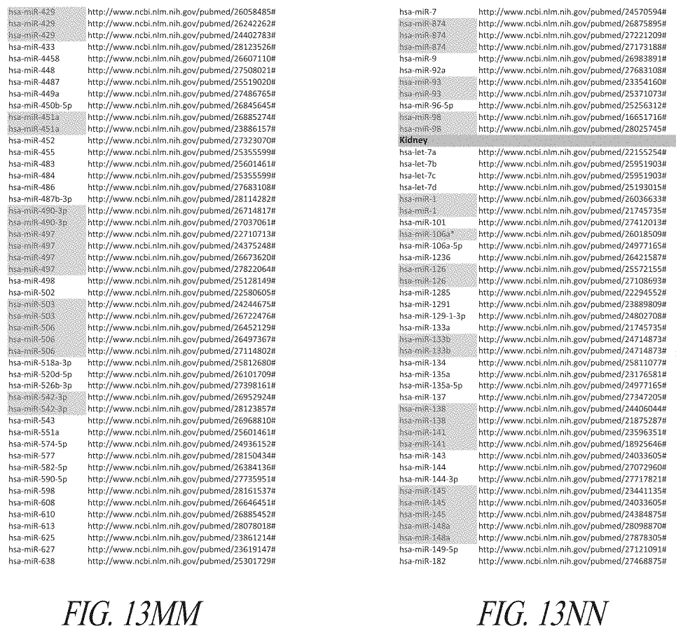

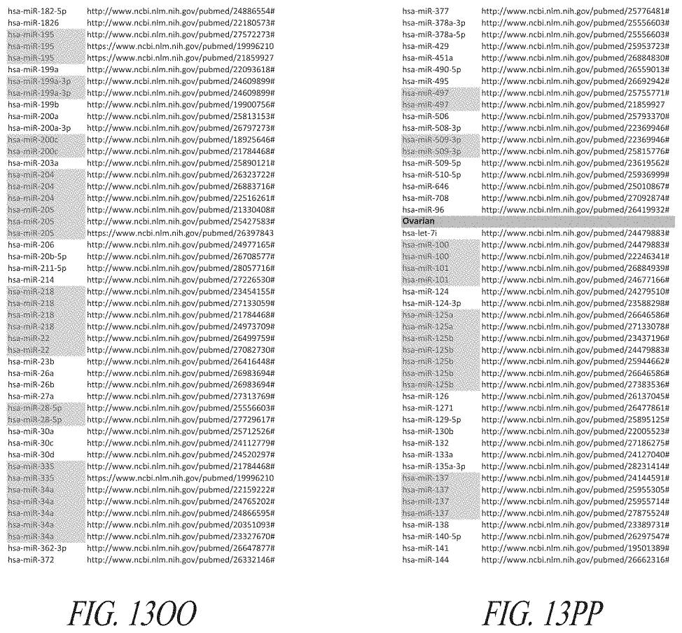

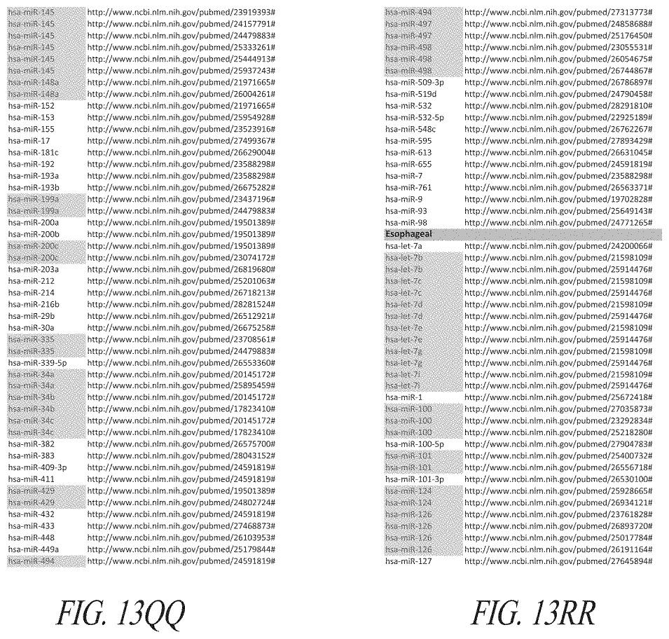

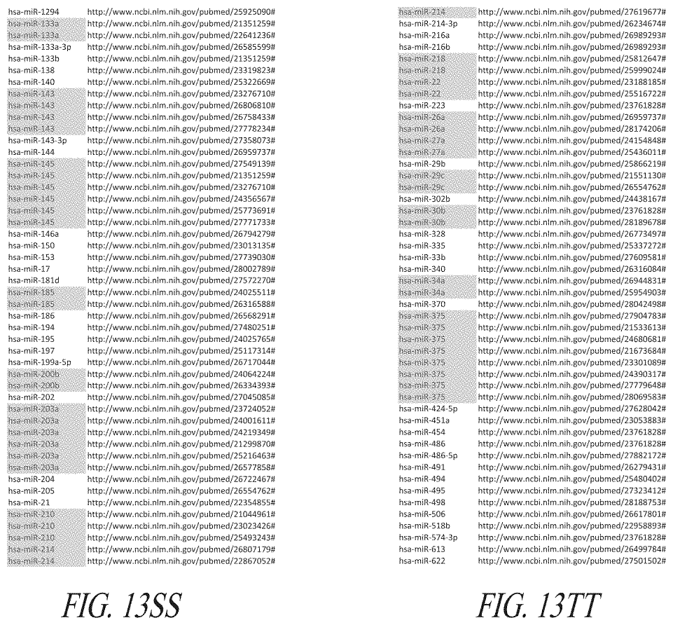

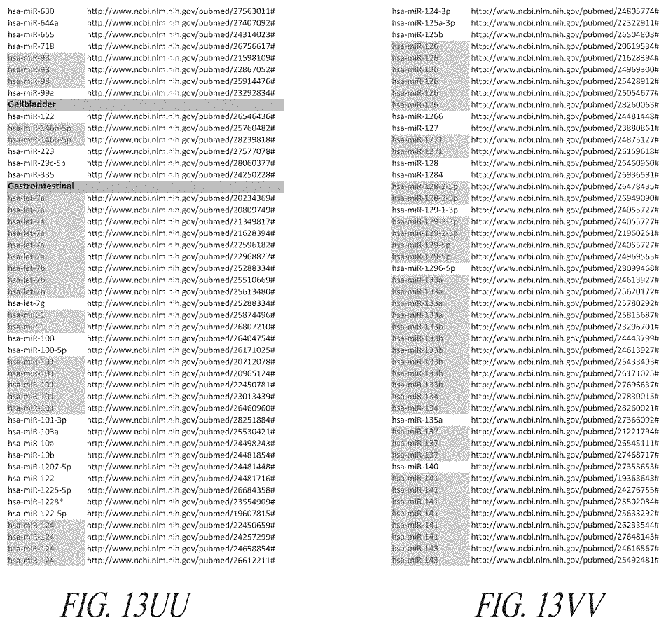

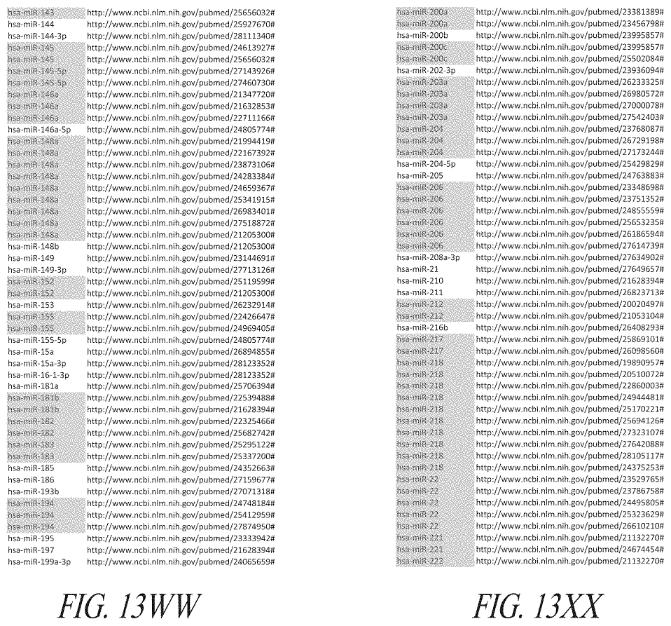

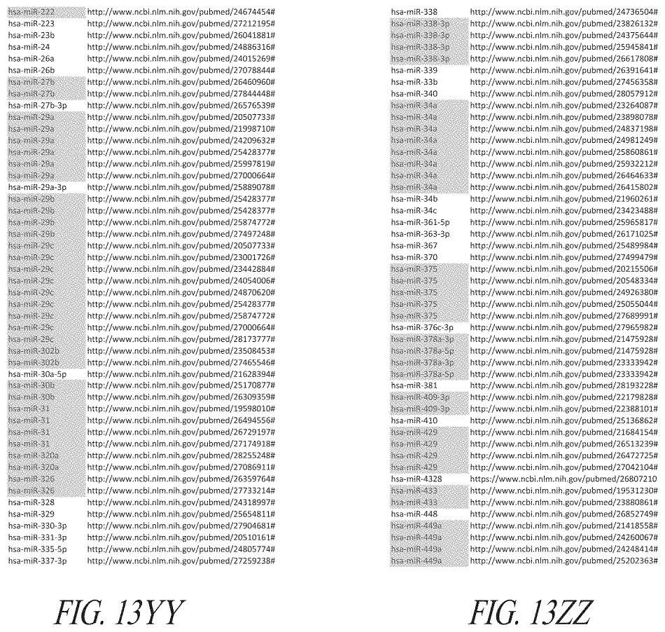

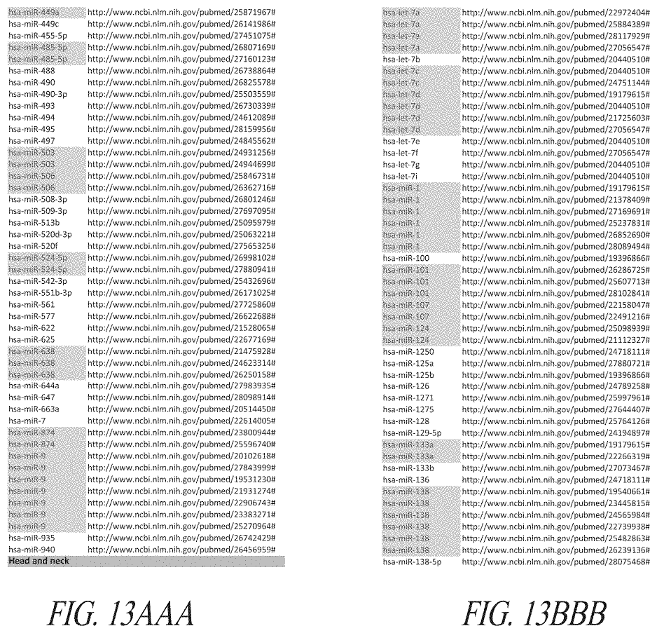

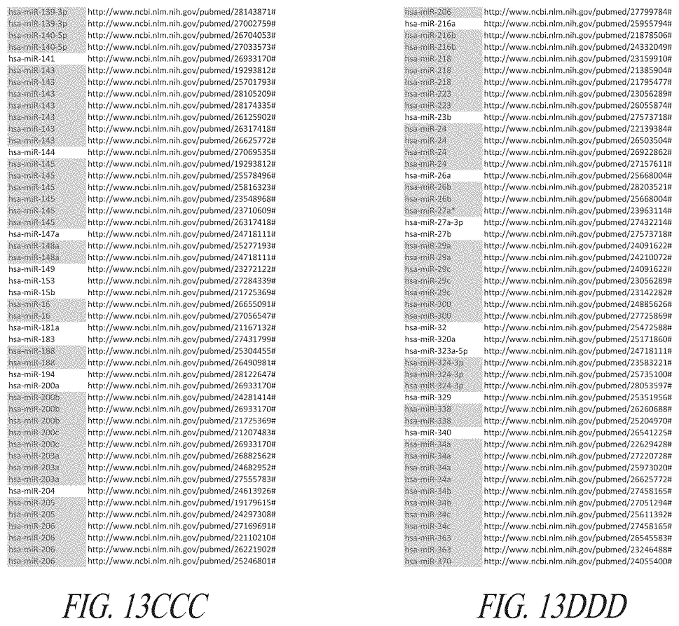

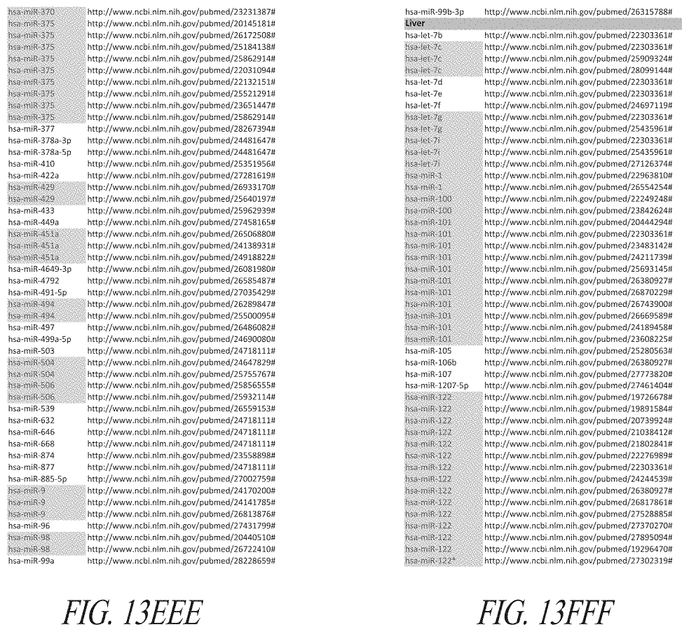

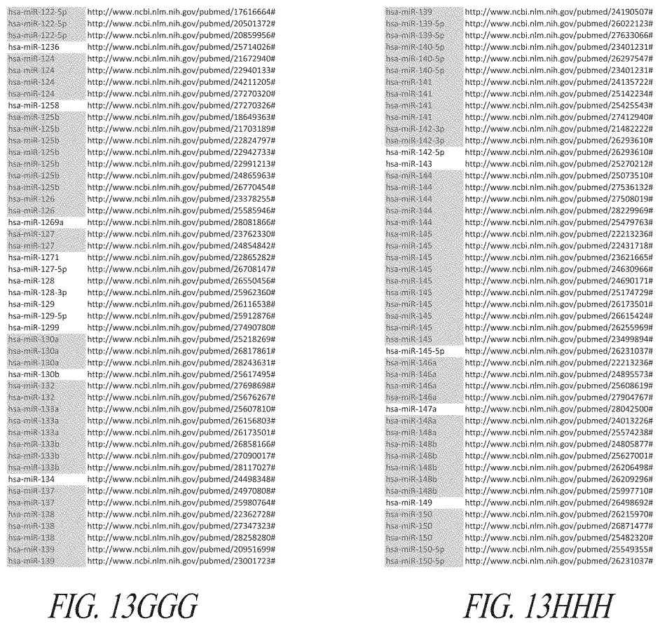

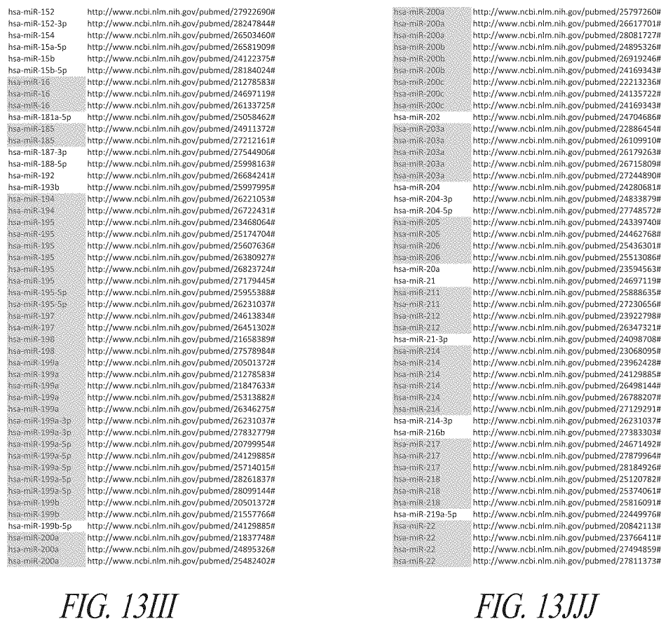

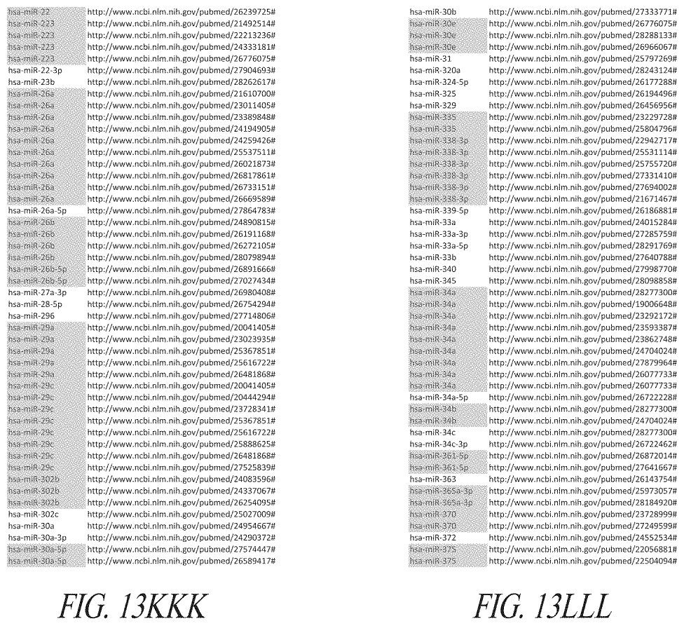

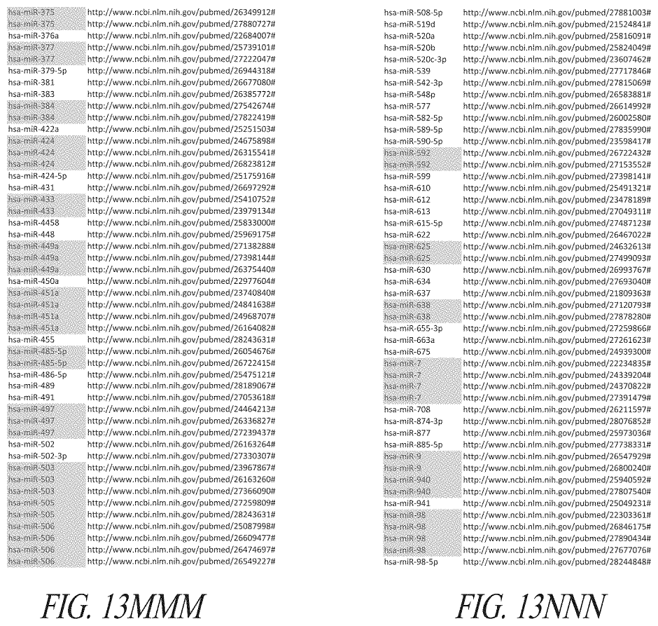

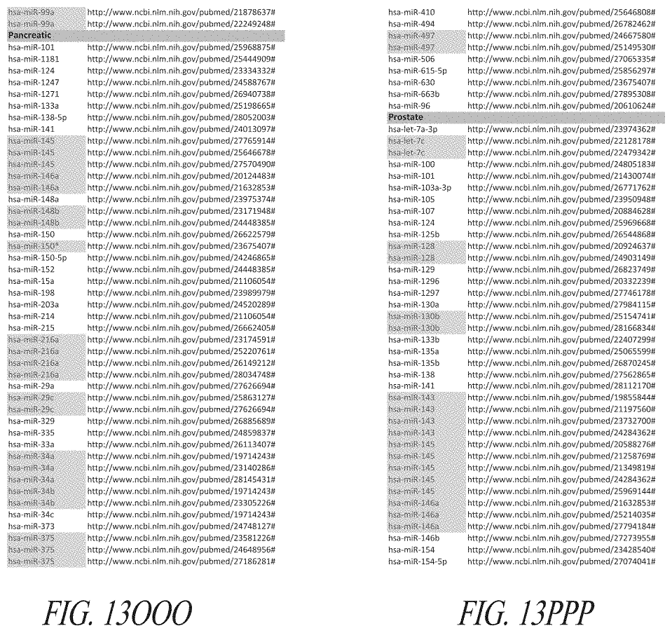

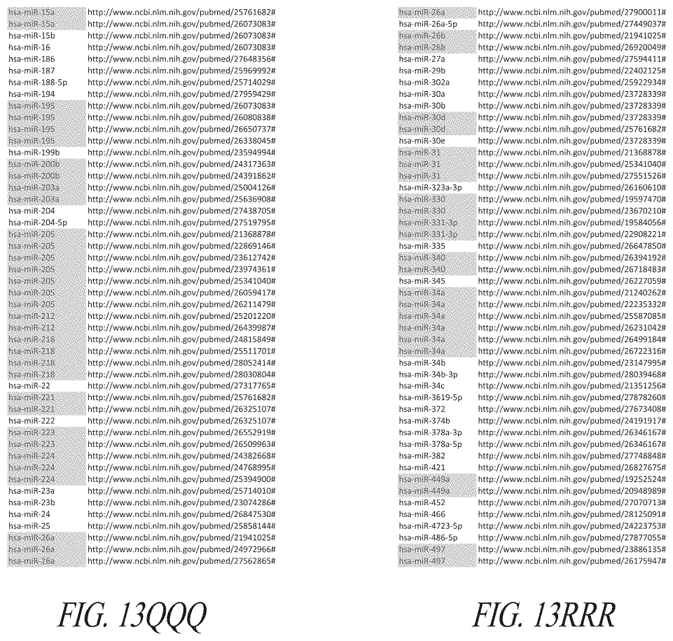

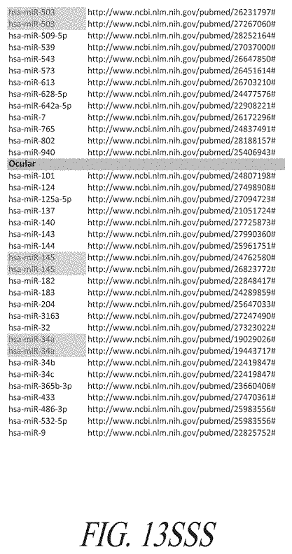

The term “microRNA” or “miRNA” as used herein refers to a family of short (typically 21-25 nucleotides), endogenous, single-stranded RNAs expressed in a wide range of organisms including both animals and plants. There are over 1000 unique miRNAs expressed in humans. miRNAs bind to specific target sequences found in messenger RNAs (mRNAs). Binding to complementary or partially complementary sequences (target sequences) in mRNA molecules results in down-regulation of gene expressing by cleavage of the mRNA, increased degradation from shortening of its polyA tail, and direct translational repression. A selected list of microRNAs in tumors (along with associated references) are provided in FIGS. 13A-13Z, 13AA-13ZZ, and 13AAA-13SSS, which list and associated references are incorporated by reference in their entirety.

The term “oncolytic herpes virus” or “oHSV” refers generally to a herpes virus capable of replicating in and killing tumor cells. Within certain embodiments the virus can be engineered in order to more selectively target tumor cells. Representative examples of oncolytic herpes viruses are described in U.S. Pat. Nos. 7,223,593, 7,537,924, 7,063,835, 7,063,851, 7,118,755, 8,216,564, 8,277,818, and 8,680,068, all of which are incorporated by reference in their entirety.

“Treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

Representative forms of cancer include carcinomas, leukemia's, lymphomas, myelomas and sarcomas. Further examples include, but are not limited to cancer of the bile duct cancer, brain (e.g., glioblastoma), breast, cervix, colorectal, CNS (e.g., acoustic neuroma, astrocytoma, craniopharyogioma, ependymoma, glioblastoma, hemangioblastoma, medulloblastoma, menangioma, neuroblastoma, oligodendroglioma, pinealoma and retinoblastoma), endometrial lining, hematopoietic cells (e.g., leukemia's and lymphomas), kidney, larynx, lung, liver, oral cavity, ovaries, pancreas, prostate, skin (e.g., melanoma and squamous cell carcinoma) and thyroid. Cancers can comprise solid tumors (e.g., sarcomas such as fibrosarcoma, myxosarcoma, liposarcoma, chondrosarcoma and osteogenic sarcoma), be diffuse (e.g., leukemia's), or some combination of these (e.g., a metastatic cancer having both solid tumors and disseminated or diffuse cancer cells). Cancers can also be resistant to conventional treatment (e.g. conventional chemotherapy and/or radiation therapy).

Particularly preferred cancers to be treated include lung tumors, breast and prostate tumors, glioblastomas, tumors of the gastro-intestinal tract (and associated organs) e.g., esophagus, cholangiocarcinoma, anal, stomach, intestine, pancreatic, colon and liver, and all surface injectable tumors (e.g., melanomas).

Benign tumors and other conditions of unwanted cell proliferation may also be treated.

In order to further an understanding of the various embodiments herein, the following sections are provided which describe various embodiments: A. Oncolytic Herpes Viruses; B. MicroRNAs; C. Therapeutic Compositions, and D. Administration

### A. Oncolytic Herpes Viruses

Herpes Simplex Virus (HSV) 1 and 2 are members of the Herpesviridae family, which infects humans. The HSV genome contains two unique regions, which are designated unique long (U_{L}) and unique short (U_{S}) region. Each of these regions is flanked by a pair of inverted terminal repeat sequences. There are about 75 known open reading frames. The viral genome has been engineered to develop oncolytic viruses for use in e.g. cancer therapy. Tumor-selective replication of HSV may be conferred by mutation of the HSV ICP34.5 (also called γ34.5) gene. HSV contains two copies of ICP34.5. Mutants inactivating one or both copies of the ICP34.5 gene are known to lack neurovirulence, i.e. be avirulent/non-neurovirulent and be oncolytic. Tumor selective replication of HSV may also be conferred by controlling expression of key viral genes such as ICP27 and/or ICP4.

Suitable oncolytic HSV may be derived from either HSV-1 or HSV-2, including any laboratory strain or clinical isolate. In some embodiments, the oHSV may be or may be derived from one of laboratory strains HSV-1 strain 17, HSV-1 strain F, or HSV-2 strain HG52. In other embodiments, it may be of or derived from non-laboratory strain JS-1. Other suitable HSV-1 viruses include HrrR3 (Goldstein and Weller, J. Virol. 62, 196-205, 1988), G2O7 (Mineta et al. Nature Medicine. 1(9):938-943, 1995; Kooby et al. The FASEB Journal, 13(11):1325-1334, 1999); G47Delta (Todo et al. Proceedings of the National Academy of Sciences. 2001; 98(11):6396-6401); HSV 1716 (Mace et al. Head & Neck, 2008; 30(8):1045-1051; Harrow et al. Gene Therapy. 2004; 11(22):1648-1658); HF10 (Nakao et al. Cancer Gene Therapy. 2011; 18(3):167-175); NV1020 (Fong et al. Molecular Therapy, 2009; 17(2):389-394); T-VEC (Andtbacka et al. Journal of Clinical Oncology, 2015: 33(25):2780-8); J100 (Gaston et al. PloS one, 2013; 8(11):e81768); M002 (Parker et al. Proceedings of the National Academy of Sciences, 2000; 97(5):2208-2213); NV1042(Passer et al. Cancer Gene Therapy. 2013; 20(1):17-24); G207-IL2 (Carew et al. Molecular Therapy, 2001; 4(3):250-256); rQNestin34.5 (Kambara et al. Cancer Research, 2005; 65(7):2832-2839); G47Δ-mIL-18 (Fukuhara et al. Cancer Research, 2005; 65(23):10663-10668); and those vectors which are disclosed in PCT applications PCT/US2017/030308 entitled “HSV Vectors with Enhanced Replication in Cancer Cells”, and PCT/US2017/018539 entitled “Compositions and Methods of Using Stat1/3 Inhibitors with Oncolytic Herpes Virus”, all of the above of which are incorporated by reference in their entirety.

The oHSV vector has at least one γ34.5 gene that is modified with miRNA target sequences in its 3′ UTR as disclosed herein; there are no unmodified γ34.5 genes in the vector. In some embodiments, the oHSV has two modified γ34.5 genes; in other embodiments, the oHSV has only one γ34.5 gene, and it is modified. In some embodiments, the modified γ34.5 gene(s) are constructed in vitro and inserted into the oHSV vector as replacements for the viral gene(s). When the modified γ34.5 gene is a replacement of only one γ34.5 gene, the other γ34.5 is deleted. Either native γ34.5 gene can be deleted. In one embodiment, the terminal repeat, which comprises γ34.5 gene and ICP4 gene, is deleted. As discussed herein, the modified γ34.5 gene may comprise additional changes, such as having an exogenous promoter.

The oHSV may have additional mutations, which may include disabling mutations e.g., deletions, substitutions, insertions), which may affect the virulence of the virus or its ability to replicate. For example, mutations may be made in any one or more of ICP6, ICPO, ICP4, ICP27, ICP47, ICP24, ICP56. Preferably, a mutation in one of these genes (optionally in both copies of the gene where appropriate) leads to an inability (or reduction of the ability) of the HSV to express the corresponding functional polypeptide. In some embodiments, the promoter of a viral gene may be substituted with a promoter that is selectively active in target cells or inducible upon delivery of an inducer or inducible upon a cellular event or particular environment.

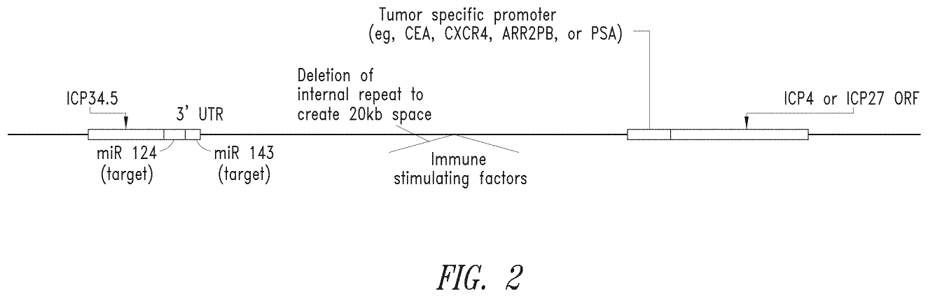

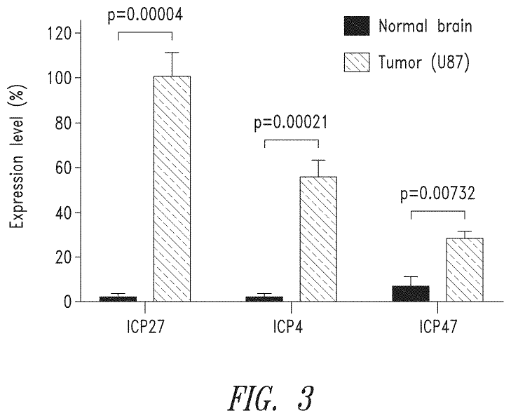

In certain embodiments the expression of ICP4 or ICP27 is controlled by an exogenous promoter, e.g., a tumor-specific promoter. Exemplary tumor-specific promoters include survivin, CEA, CXCR4, PSA, ARR2PB, or telomerase; other suitable tumor-specific promoters may be specific to a single tumor type and are known in the art. Other elements may be present. In some cases, an enhancer such as NFkB/oct4/sox2 enhancer is present. As well, the 5′UTR may be exogenous, such as a 5′UTR from growth factor genes such as FGF. See FIG. 2 for an exemplary construct.

The oHSV may also have genes and nucleotide sequences that are non-HSV in origin. For example, a sequence that encodes a prodrug, a sequence that encodes a cytokine or other immune stimulating factor, a tumor-specific promoter, an inducible promoter, an enhancer, a sequence homologous to a host cell, among others may be in the oHSV genome. Exemplary sequences encode IL12, IL15, IL15 receptor alpha subunit, OX40L, PD-L1 blocker or a PD-1 blocker. For sequences that encode a product, they are operatively linked to a promoter sequence and other regulatory sequences (e.g., enhancer, polyadenylation signal sequence) necessary or desirable for expression.

The regulatory region of viral genes may be modified to comprise response elements that affect expression. Exemplary response elements include response elements for NF-κB, Oct-3/4-SOX2, enhancers, silencers, cAMP response elements, CAAT enhancer binding sequences, and insulators. Other response elements may also be included. A viral promoter may be replaced with a different promoter. The choice of the promoter will depend upon a number of factors, such as the proposed use of the HSV vector, treatment of the patient, disease state or condition, and ease of applying an inducer (for an inducible promoter). For treatment of cancer, generally when a promoter is replaced it will be with a cell-specific or tissue-specific or tumor-specific promoter. Tumor-specific, cell-specific and tissue-specific promoters are known in the art. Other gene elements may be modified as well. For example, the 5′ UTR of the viral gene may be replaced with an exogenous UTR.

### B. MicroRNAs

As noted above, the present invention provides oHSVs having at least two miRNA target sequences. Briefly, miRNA binds to its target sequence in an mRNA, which is typically in the 3′-untranslated region (3′-UTR). Binding may initiate or require a region called the “seed region” located from about nucleotides 2-8 from the 5′-end of the miRNA. When there is partial complementarity, the 5′-end tends to have more identity to the target sequence than the 3′-end. Higher amount of complementarity may enhance repression of the mRNA, especially through mRNA cleavage.

Individual miRNAs and groups of miRNAs may be expressed exclusively or preferentially in certain tissue types. miRNAs that are enriched or exclusive to neuronal cells include of mIR-122, miR-124, miR-124*, miR-127, miR-128, miR-129, miR-129*, miR-132, mIR-133a, mIR133b, miR-135b, miR-136, miR-136*, miR-137, miR-139-5p, miR-143, mIR-145, miR-154, miR-184, miR-188, miR-204, mIR216a, miR-299, miR-300-3p, miR-300-5p, miR-323, miR-329, miR-337, miR-335, miR-341, miR-369-3p, miR-369-5p, miR-376a, miR-376a*, miR-376b-3p, miR-376b-5p, miR-376c, miR-377, miR-379, miR-379*, miR-382, miR-382*, miR-409-5p, miR-410, miR-411, miR-431, miR-433, miR-434, miR-451, miR-466b, miR-485, miR-495, miR-539, miR-541, miR-543*, miR-551b, miR-758, and miR-873. By convention, the strand that is more frequently found to be the final product is referred to as miRNA and the rarer partner as miRNA*. A selected list of microRNAs in tumors (along with associated references) are provided in FIGS. 13A-13Z, 13AA-13ZZ, and 13AAA-13SSS, which list and associated references are incorporated by reference in their entirety.

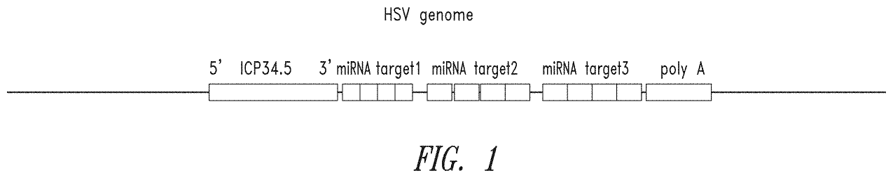

The miRNA target sequences are inserted in the 3′UTR of the γ34.5 gene. There are at least two miRNA target sequences that are inserted in tandem. There may be at least three, at least four, at least five, at least six, at least 10, and so on target sequences. Within other embodiments there are less than 10, 20, 50, or 100 target sequences. An optimal number of target sequences can be determined by assaying expression levels of ICP34.5. A low to nonexistent level of ICP34.5 is desired. The multiple miRNA target sequences may all bind the same miRNA or may bind different miRNAs. The target sequences may be in clusters (e.g., FIG. 1) in which for example, there are at least two target sequences in tandem that bind a first miRNA followed by at least two target sequences in tandem that bind a second miRNA followed by at least two target sequences that bind a third miRNA. Alternatively, the multiple miRNA target sequences that bind different miRNAs may be in no particular order. As well, there may be only a single copy of each miRNA target sequence. In some embodiments, there are 3-5 different miRNA targets. In other embodiments, there are 3-5 copies of each target sequence. In other embodiments, there are 3-5 different miRNA targets, and 3-5 copies of each of these target sequences in clusters. See FIG. 1 for an exemplary construct.

The multiple miRNA target sequences may be adjacent without intervening nucleotides or have from 1 to about 25, or from 1 to about 20, or from 1 to about 15, or from 1 to about 10, or from 1 to about 5, or from 3 to about 10, or from 5 to about 10 intervening nucleotides. Intervening nucleotides may be chosen to have a similar G+C content as the 3′UTR and preferably do not contain a polyadenylation signal sequence. Other considerations for choosing the intervening nucleotides are known in the art.

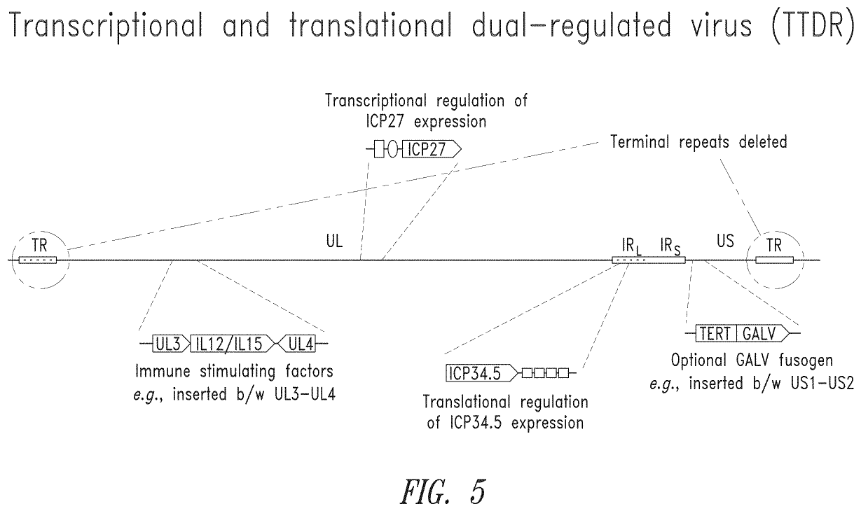

Within certain embodiments of the invention oHSV as described herein are constructed to employ both transcriptional and translational dual-regulation (also referred to as “TTDR”). One exemplary illustration of such vectors is provided in FIG. 5. Briefly, within certain preferred embodiments translational control of the ICP34.5 gene is obtained by inserting five copies of the binding sites for miR-124 and miR-143 in the 3′-UTR of the ICP34.5 gene. Key elements of the platform virus vector may also include transcriptional control of the ICP27 gene, a gene essential for viral replication, using a tumor-specific promoter.

A wide variety of HSV-1 strains may be used as the backbone for construction of recombinant oncolytic viruses, including strain 17, strain KOS, strain F, and strain McKrae. All viral mutagenesis may be performed in Escherichia coli using standard lambda Red-mediated recombineering techniques implemented on the HSV-1 genome cloned into a bacterial artificial chromosome (BAC) (see generally: Tischer B K, Smith G A, Osterrieder N. Methods Mol Biol. 2010; 634:421-30. doi: 10.1007/978-1-60761-652-8_30. PMID: 20677001; Tischer B K, von Einem J, Kaufer B, and Osterrieder N., BioTechniques 40:191-197, February 2006 (including the Supplementary Material, doi: 10.2144/000112096; and Tischer B K, Smith, G A and Osterrieder N. Chapter 30, Jeff Braman (ed.), In Vitro Mutagenesis Protocols: Third Edition, Methods in Molecular Biology, vol. 634, doi: 10.1007/978-1-60761-652-8_30, Springer Sceince+Business Media, LLC 2010).

Tumor-specific promoters may also be used to drive expression of a cassette encoding the immunomodulators IL12/IL15/IL15RA, which boost the anti-tumor immune response. The immunomodulator expression cassette may be controlled by a hCEA, hCXCR4, or PSA promoter and be inserted into the viral genome in a location which does not have a negative impact on viral gene expression and replication, such as between viral genes US1/US2, UL3/UL4 and /or UL50/UL51. To facilitate in vivo testing in a variety of mouse models, other recombinant viruses may be constructed expressing murine IL12 instead of human IL12. Human IL15 can be retained in mouse-specific oncolytic viruses due to its activity in mouse cells.

The vectors may include an expression cassette encoding a fusogenic form of the Gibbon ape leukemia virus (GALV) env protein lacking the C-terminal R-peptide, which enhances virus cytotoxicity. Within other embodiments the expression cassette can encode a fusogenic form of HSV-1 glycoprotein B. Within certain preferred embodiments, glycoprotein B can be truncated (e.g., with a deletion occurring after amino acid 876 of gB (“gB-876t”). The cassette may be inserted into the viral genome in a location which does not have a negative impact on viral gene expression and replication such as between viral genes US1/US2, UL3/UL4 and /or UL50/UL51.

BAC recombineering requires the presence of exogenous BAC DNA within the viral genome to facilitate mutagenesis in E. coli. The BAC sequence is most commonly inserted either between viral genes such as US1/US2, UL3/UL4 and /or UL50/UL51, or, into the thymidine kinase (TK) gene, which can disrupt expression of native TK. TK-deficient viral vectors may include an expression cassette for the HSV-1 thymidine kinase (TK) gene under the control of a constitutive promoter inserted into a non-coding region of the viral genome. Presence of the exogenous TK gene enhances virus safety by rendering the virus sensitive to common treatment with guanosine analogues, such as ganciclovir and acyclovir.

Within alternative embodiments, the originally disrupted TK may be recovered instead of inserting another TK, or, the TK gene may be disrupted and not replaced or recovered at all in order to further reduce neurovirule (since TK-null virus cannot reactivate from latency). Even if TK is disrupted, the virus would still be sensitive to treatment with drugs that are not dependent on TK for their function. For example, foscarnet and cidofovir inhibit viral DNA polymerase and are not TK dependent.

The promoter driving expression of the key HSV-1 transcriptional regulator ICP27 may be replaced with a tumor-specific promoter such as hCEA, hCXCR4, PSA, or Probasin (ARR2PB). The 3′ UTR of the viral gene encoding the neurovirulence factor ICP34.5 may also be modified by insertion of multiple copies of microRNA recognition elements to abrogate production of ICP34.5 in tissues containing high levels of the corresponding microRNA. In an exemplary embodiment, five copies of miR-124 and five copies of miR-143 recognition elements may be inserted in tandem into the 3′ UTR of ICP34.5.

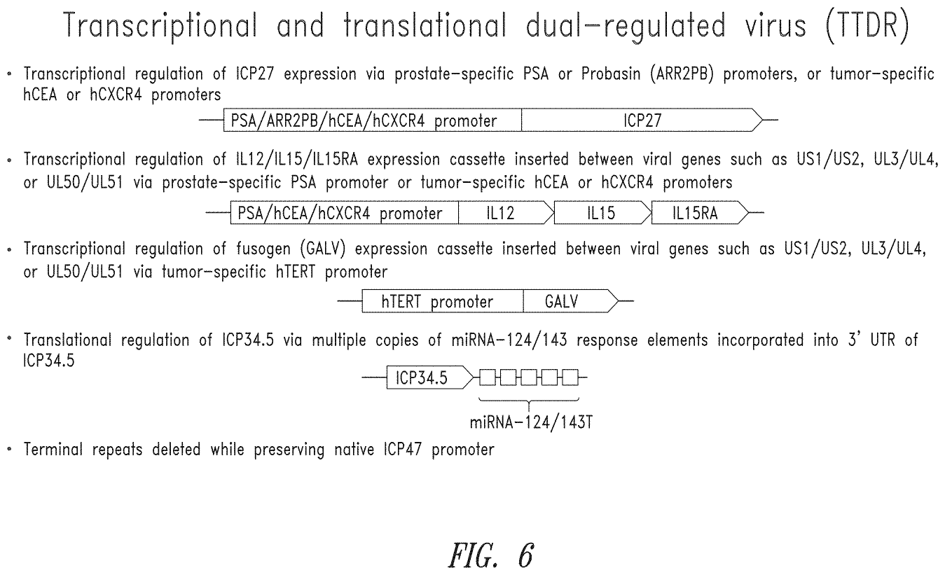

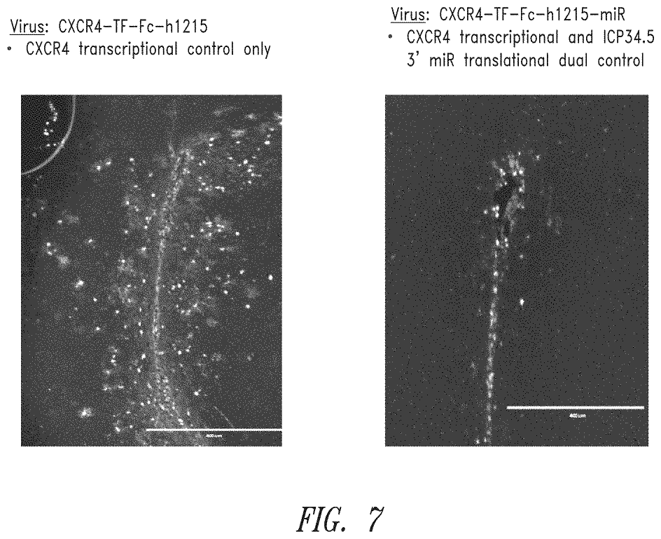

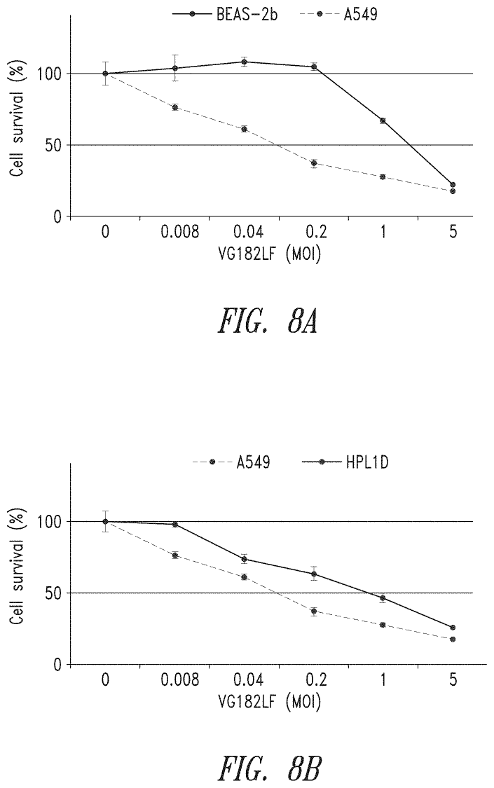

The terminal repeat region of the viral genome may be completely deleted to reduce the overall genome size and create more space for transgene insertions; the deleted TR is engineered to avoid disrupting the native promoter of the ICP47 gene, which is normally part of the terminal repeats. A similar modification may be carried out by deleting the internal repeat region instead of the terminal repeat region. Further details of exemplary elements discussed herein are illustrated in FIG. 6.

### C. Therapeutic Compositions

Therapeutic compositions are provided that may be used to prevent, treat, or ameliorate the effects of a disease, such as, for example, cancer. More particularly, therapeutic compositions are provided comprising at least one oncolytic virus as described herein.

In certain embodiments, the compositions will further comprise a pharmaceutically acceptable carrier. The phrase “pharmaceutically acceptable carrier” is meant to encompass any carrier, diluent or excipient that does not interfere with the effectiveness of the biological activity of the oncolytic virus and that is not toxic to the subject to whom it is administered (see generally Remington: The Science and Practice of Pharmacy, Lippincott Williams & Wilkins; 21st ed. (May 1, 2005 and in The United States PharmacopE1A: The National Formulary (USP 40-NF 35 and Supplements).

In the case of an oncolytic virus as described herein, non-limiting examples of suitable pharmaceutical carriers include phosphate buffered saline solutions, water, emulsions (such as oil/water emulsions), various types of wetting agents, sterile solutions, and others. Additional pharmaceutically acceptable carriers include gels, bioabsorbable matrix materials, implantation elements containing the oncolytic virus, or any other suitable vehicle, delivery or dispensing means or material(s). Such carriers can be formulated by conventional methods and can be administered to the subject at an effective dose. Additional pharmaceutically acceptable excipients include, but are not limited to, water, saline, polyethylene glycol, hyaluronic acid and ethanol. Pharmaceutically acceptable salts can also be included therein, e.g., mineral acid salts (such as hydrochlorides, hydrobromides, phosphates, sulfates, and the like) and the salts of organic acids (such as acetates, propionates, malonates, benzoates, and the like). Such pharmaceutically acceptable (pharmaceutical-grade) carriers, diluents and excipients that may be used to deliver the oHSV to a cancer cell will preferably not induce an immune response in the individual (subject) receiving the composition (and will preferably be administered without undue toxicity).

The compositions provided herein can be provided at a variety of concentrations. For example, dosages of oncolytic virus can be provided which ranges from about 10⁶ to about 10⁹ pfu. Within further embodiments, the dosage can range from about 10⁶ to about 10⁸ pfu/ml, with up to 4 mls being injected into a patient with large lesions (e.g., >5 cm) and smaller amounts (e.g., up to 0.1mls) in patients with small lesions (e.g., <0.5 cm) every 2-3 weeks, of treatment.

Within certain embodiments of the invention, lower dosages than standard may be utilized. Hence, within certain embodiments less than about 10⁶ pfu/ml (with up to 4 mls being injected into a patient every 2-3 weeks) can be administered to a patient.

The compositions may be stored at a temperature conducive to stable shelf-life and includes room temperature (about 20° C.), 4° C., −20° C., −80° C., and in liquid N2. Because compositions intended for use in vivo generally don't have preservatives, storage will generally be at colder temperatures. Compositions may be stored dry (e.g., lyophilized) or in liquid form.

### D. Administration

In addition to the compositions described herein, various methods of using such compositions to treat or ameliorate cancer are provided, comprising the step of administering an effective dose or amount of oHSV as described herein to a subject.

The terms “effective dose” and “effective amount” refers to amounts of the oncolytic virus that is sufficient to effect treatment of a targeted cancer, e.g., amounts that are effective to reduce a targeted tumor size or load, or otherwise hinder the growth rate of targeted tumor cells. More particularly, such terms refer to amounts of oncolytic virus that is effective, at the necessary dosages and periods of treatment, to achieve a desired result. For example, in the context of treating a cancer, an effective amount of the compositions described herein is an amount that induces remission, reduces tumor burden, and/or prevents tumor spread or growth of the cancer. Effective amounts may vary according to factors such as the subject's disease state, age, gender, and weight, as well as the pharmaceutical formulation, the route of administration, and the like, but can nevertheless be routinely determined by one skilled in the art.

The therapeutic compositions are administered to a subject diagnosed with cancer or is suspected of having a cancer. Subjects may be human or non-human animals.

The compositions are used to treat cancer. The terms “treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

Representative forms of cancer include carcinomas, leukemia's, lymphomas, myelomas and sarcomas. Further examples include, but are not limited to cancer of the bile duct, brain (e.g., glioblastoma), breast, cervix, colorectal, CNS (e.g., acoustic neuroma, astrocytoma, craniopharyogioma, ependymoma, glioblastoma, hemangioblastoma, medulloblastoma, menangioma, neuroblastoma, oligodendroglioma, pinealoma and retinoblastoma), endometrial lining, hematopoietic cells (e.g., leukemia's and lymphomas), kidney, larynx, lung, liver, oral cavity, ovaries, pancreas, prostate, skin (e.g., melanoma and squamous cell carcinoma), GI (e.g., esophagus, stomach, and colon) and thyroid. Cancers can comprise solid tumors (e.g., sarcomas such as fibrosarcoma, myxosarcoma, liposarcoma, chondrosarcoma and osteogenic sarcoma), be diffuse (e.g., leukemia's), or some combination of these (e.g., a metastatic cancer having both solid tumors and disseminated or diffuse cancer cells). Cancers can also be resistant to conventional treatment (e.g. conventional chemotherapy and/or radiation therapy).

Particularly preferred cancers to be treated include lung tumors, breast and prostate tumors, glioblastomas, tumors of the gastro-intestinal tract (and associated organs) e.g., esophagus, cholangiocarcinoma, anal, stomach, intestine, pancreatic, colon and liver, and all surface injectable tumors (e.g., melanomas). Benign tumors and other conditions of unwanted cell proliferation may also be treated.

The recombinant herpes simplex viruses described herein may be given by a route that is e.g. oral, topical, parenteral, systemic, intravenous, intramuscular, intraocular, intrathecal, intratumor, subcutaneous, or transdermal. Within certain embodiments the oncolytic virus may be delivered by a cannula, by a catheter, or by direct injection. The site of administration may be intra-tumor or at a site distant from the tumor. The route of administration will often depend on the type of cancer being targeted.

The optimal or appropriate dosage regimen of the oncolytic virus is readily determinable within the skill of the art, by the attending physician based on patient data, patient observations, and various clinical factors, including for example a subject's size, body surface area, age, gender, and the particular oncolytic virus being administered, the time and route of administration, the type of cancer being treated, the general health of the patient, and other drug therapies to which the patient is being subjected. According to certain embodiments, treatment of a subject using the oncolytic virus described herein may be combined with additional types of therapy, such as chemotherapy using, e.g., a chemotherapeutic agent such as etoposide, ifosfamide, adriamycin, vincristine, doxycycline, and others.

Recombinant herpes simplex viruses described herein may be formulated as medicaments and pharmaceutical compositions for clinical use and may be combined with a pharmaceutically acceptable carrier, diluent, excipient or adjuvant. The formulation will depend, at least in part, on the route of administration. Suitable formulations may comprise the virus and inhibitor in a sterile medium. The formulations can be fluid, gel, paste or solid forms. Formulations may be provided to a subject or medical professional

A therapeutically effective amount is preferably administered. This is an amount that is sufficient to show benefit to the subject. The actual amount administered and time-course of administration will depend at least in part on the nature of the cancer, the condition of the subject, site of delivery, and other factors.

Within yet other embodiments of the invention the oncolytic virus can be administered by a variety of methods, e.g., intratumorally, intravenously, or, after surgical resection of a tumor.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

The following are additional exemplary embodiments of the present disclosure:

1) An recombinant herpes simplex virus comprising at least one ICP34.5 gene having at least two miRNA target sequences in the 3′ untranslated region of ICP34.5. Within related embodiments a recombinant herpes simplex virus is provided comprising a modified oncolytic herpes virus genome, wherein the modified herpes virus genome comprises at least one miRNA target sequence operably linked to a first, or, to a first and a second copy of an ICP34.5 gene.

2) The recombinant herpes simplex virus of embodiment 1, wherein the at least two miRNA target sequences are targets for the same miRNA.

3) The recombinant herpes simplex virus of either of embodiment 1 or 2, wherein the at least two miRNA target sequences are targets for an miRNA selected from the group consisting of mIR-122, miR-124, miR-124*, miR-127, miR-128, miR-129, miR-129*, miR-132, mIR-133a, mIR133b, miR-135b, miR-136, miR-136*, miR-137, miR-139-5p, miR-143, mIR-145, miR-154, miR-184, miR-188, miR-204, mIR216a, miR-299, miR-300-3p, miR-300-5p, miR-323, miR-329, miR-337, miR-335, miR-341, miR-369-3p, miR-369-5p, miR-376a, miR-376a*, miR-376b-3p, miR-376b-5p, miR-376c, miR-377, miR-379, miR-379*, miR-382, miR-382*, miR-409-5p, miR-410, miR-411, miR-431, miR-433, miR-434, miR-451, miR-466b, miR-485, miR-495, miR-539, miR-541, miR-543*, miR-551b, miR-758, and miR-873. By convention, the strand that is more frequently found to be the final product is referred to as miRNA and the rarer partner as miRNA*. Within certain embodiments of the invention recombinant herpes simplex viruses are provided according to embodiments 1, 2, or 3, wherein the miRNA target sites comprise one, two, three, four, five, six or more copies of the binding sites for miR-124 and miR-143.

4) The recombinant herpes simplex virus of any of embodiments 1-3, further comprising a modified ICP27 or ICP4 gene, wherein the modification is a replacement of the 5′UTR. Within other embodiments, the ICP27, or, ICP4 gene is modified by replacement of the native promoter. Within particularly preferred embodiments of the invention, ICP27 is modified by replacement of the native promoter with an hCEA promoter, or, a hCXCR4 promoter.

5) The recombinant herpes simplex virus of any of embodiments 1, 2, 3, or, 4 further comprising a modified ICP27, wherein the modification is replacement of the entire promoter-regulatory region of ICP27. Within further embodiments of the above, the herpes simplex virus is HSV-1. Within yet further embodiments of any one of embodiments 1, 2, 3, or 4, the recombinant herpes simplex virus further comprises a fusogenic mutation in a gene encoding for glycoprotein B (gB). Within related embodiments, the gene encoding glycoprotein B (gB) encodes a glycoproptein B variant that terminates after amino acid 876. Within yet other embodiments a recombinant herpes simplex virus according to any one of embodiments 1, 2, 3, or, 4 is provided wherein the genome further comprises a modified gene encoding for glycoprotein B (gB), wherein the modified gene encodes for a glycoprotein B variant that terminates after amino acid 876. Within yet further embodiments, the recombinant herpes simplex virus comprises additional mutations or modifications in at least one viral gene selected from the group consisting of ICP6, ICP0, ICP4, ICP27, ICP47, ICP 24, and ICP56. Within certain preferred embodiments the additional mutations or modifications are in non-coding regions of the viral genes.

6) The recombinant herpes simplex virus of any of embodiments 1, 2, 3, 4, or, 5, further comprising gene sequences encoding at least one immunostimulatory factor. Representative immunostimulatory factors include IL12, IL15, IL15 receptor alpha subunit, OX40L, and a PD-L1 blocker.

7) The recombinant herpes simplex virus of any of embodiments 1, 2, 3, 4, 5, or, 6, further comprising a gene sequence encoding an immune stimulating factor, or a checkpoint blocking peptide. Within further aspects of embodiments 1, 2, 3, 4, or, 5, the recombinant herpes simplex virus further comprises at least one nucleic acid encoding a non-viral protein selected from the group consisting of immunostimulatory factors, antibodies, and checkpoint blocking peptides. Within related embodiments the at least one nucleic acid is operably linked to a tumor-specific promoter.

8) A method of treating cancer, comprising administering the recombinant herpes simplex virus of any of embodiments 1-7. Particularly preferred cancers to be treated include lung tumors, breast and prostate tumors, glioblastomas, tumors of the gastro-intestinal tract (and associated organs) e.g., esophagus, cholangiocarcinoma, anal, stomach, intestine, pancreatic, colon and liver, and all surface injectable tumors (e.g., melanomas).

The following are yet further embodiments of the invention:

9) A recombinant herpes simplex virus comprising a modified oncolytic herpes virus genome, wherein the modified herpes virus genome comprises at least one miRNA target sequence operably linked to a first or to a first and a second copy of an ICP34.5 gene. Within preferred embodiments the herpes simplex virus produces significantly reduced levels of functional ICP34.5 protein in untransformed cells as compared to tumor cells.

10) The recombinant herpes simplex virus of embodiment 9, wherein the second copy of the ICP34.5 gene comprises an inactivating mutation.

11) The recombinant herpes simplex virus of embodiment 9, comprising from two to ten miRNA target sequences operably linked to the first or to the first and the second copies of the ICP34.5 gene.

12) The recombinant herpes simplex virus of embodiments 10 or 11, comprising two miRNA target sequences operably linked to the first or to the first and the second copies of the ICP34.5 gene.

13) The recombinant herpes simplex virus of embodiments 10 or 11, wherein the miRNA target sequences are inserted into a 3′ untranslated region of the first or the first and the second copies of the ICP34.5 gene.

14) The recombinant herpes simplex virus of embodiment 13, wherein the miRNA target sequences are inserted in tandem into the 3′ untranslated region.

15) The recombinant herpes simplex virus of embodiments 10 or 11, wherein the from two to ten miRNA target sequences bind a single miRNA.

16) The recombinant herpes simplex virus of embodiments 10 or 11, wherein the from two to ten miRNA target sequences bind at least two different miRNAs.

17) The recombinant herpes simplex virus or embodiment 15 or 16, wherein the miRNAs are selected from the group consisting of mIR-122, miR-124, miR-124*, miR-127, miR-128, miR-129, miR-129*, miR-132, mIR-133a, mIR133b, miR-135b, miR-136, miR-136*, miR-137, miR-139-5p, miR-143, mIR-145, miR-154, miR-184, miR-188, miR-204, mIR216a, miR-299, miR-300-3p, miR-300-5p, miR-323, miR-329, miR-337, miR-335, miR-341, miR-369-3p, miR-369-5p, miR-376a, miR-376a*, miR-376b-3p, miR-376b-5p, miR-376c, miR-377, miR-379, miR-379*, miR-382, miR-382*, mmiR-409-5p, miR-410, miR-411, miR-431, miR-433, miR-434, miR-451, miR-466b, miR-485, miR-495, miR-539, miR-541, miR-543*, miR-551b, miR-758, and miR-873. By convention, the strand that is more frequently found to be the final product is referred to as miRNA and the rarer partner as miRNA*.

18) The recombinant herpes simplex virus of embodiment 17, wherein the miRNA target sites comprise five copies of the binding sites for miR-124 and miR-143.

19) The recombinant herpes simplex virus of embodiment 9, wherein the oncolytic herpes virus is HSV-1.

20) The recombinant herpes simplex virus of embodiment 9, wherein the modified herpes virus genome comprises additional mutations or modifications in at least one viral gene selected from the group consisting of ICP6, ICP0, ICP4, ICP27, ICP47, ICP 24, and ICP56. Within preferred embodiments, the coding sequence is left intact, and said viral gene is modified by replacing the native promoter with a tumor-specific promoter.

21) The recombinant herpes simplex virus of embodiment 20, wherein the additional mutations or modifications affect the virulence of the virus or its ability to replicate.

22) The recombinant herpes simplex virus of embodiment 20, wherein the mutated or modified viral genes are ICP4 and/or ICP27.

23) The recombinant herpes simplex virus of embodiment 22, wherein the mutation or modification comprises operable linkage of the ICP4 and ICP27 genes to an exogenous 5′ untranslated region.

24) The recombinant herpes simplex virus of embodiment 23, further comprising a modified ICP27 or ICP4 gene, wherein the modification is a replacement of the 5′UTR.

25) The recombinant herpes simplex virus of embodiment 22, further comprising a modified ICP27, wherein the modification is replacement of the entire promoter-regulatory region of ICP27. Within certain embodiments the ICP27 promoter is replaced with a hCEA or hCXCR4 promoter. Within certain embodiments, only a portion of the promoter region is replaced, and the native 5′UTR is retained.

26) The recombinant herpes simplex virus of embodiment 25, further comprising at least one nucleic acid encoding a non-viral protein selected from the group consisting of immunostimulatory factors, antibodies, and checkpoint blocking peptides, wherein the at least one nucleic acid is operably linked to a tumor-specific promoter.

27) The recombinant herpes simplex virus of embodiment 26, wherein the non-viral protein is selected from the group consisting of IL12, IL15, IL15 receptor alpha subunit, OX40L, and a PD-L1 blocker.

28) The recombinant herpes simplex virus of any one of embodiments 1 to 27, further comprising an expression cassette having a nucleic acid sequence encoding a fusogenic variant of the Gibbon ape leukemia virus env protein lacking a C-terminal R-peptide and, optionally, a nucleic acid encoding HSV-1 thymidine kinase. Within other embodiments, a recombinant herpes simplex virus according to any one of embodiments 1 to 27 is provided comprising an expression cassette having a nucleic acid sequence encoding a fusogenic form of HSV-1 glycoprotein B. Within certain preferred embodiments, glycoprotein B can be truncated (e.g., with a deletion occurring after amino acid 876 of gB.

29) The recombinant herpes simplex virus of any one of embodiments 1 to 28, wherein at least one internal or terminal repeat region of the viral genome is deleted. Within certain further embodiments, the recombinant herpes virus of any one of embodiments 1 to 28 has 5× miR-124 and 5× miR-143 binding sites in the 3′UTR of ICP34.5, with terminal repeats deleted (which also deletes the second copy of ICP0, ICP4, and ICP34.5).

30) A method for lysing tumor cells, comprising providing a therapeutically effective amount of a recombinant herpes simplex virus of any of the above embodiments 1 to 29.

31) A therapeutic composition comprising a recombinant herpes simplex virus of any of the above embodiments 1 to 29 and a pharmaceutically acceptable carrier.

32) A method for treating cancer in a patient suffering therefrom, comprising the step of administering a therapeutically effective amount of the composition of embodiment 31. Particularly preferred cancers to be treated include lung tumors, breast and prostate tumors, glioblastomas, tumors of the gastro-intestinal tract (and associated organs) e.g., esophagus, cholangiocarcinoma, anal, stomach, intestine, pancreatic, colon and liver, and all surface injectable tumors (e.g., melanomas).

### EXAMPLES

### Example 1

### Hsv-1 Immediate-Early Gene Expression in Normal Mouse Brain and Human Brain Tumor U87

In this Example, HSV-1 immediate-early gene expression was compared between normal mouse brain and human brain tumor U87 at 24 hours after injection with microRNA-regulated virus. Five nude mice without tumors and five nude mice bearing human U87 brain tumors within the cranial cavity were injected once intracranially with a total of 1×10̂6 PFU/mouse of either a CXCR4-miR virus or a control CXCR4 virus. The CXCR4-miR virus is engineered to insert five miR-124/143 binding sites in tandem within the 3′ UTR of ICP34.5 as well as to modify the viral ICP27 gene such that the native ICP27 promoter-regulatory region is replaced with the tumor-specific CXCR4 promoter. The construct also includes an expression cassette for secretable IL12/1L15/1L15RA and an expression cassette for a secretable peptide which inhibits the binding of PD-1 to PD-L1. The CXCR4 virus contains a wild-type ICP34.5 gene lacking the microRNA binding sites, but is otherwise identical to the CXCR4-miR virus.

Expression of viral immediate-early genes ICP27, ICP4, and ICP47 in normal brain tissue and in tumor tissue was measured using RT-qPCR at 24 h post infection. Changes in gene expression levels for CXCR4-miR virus were determined by comparison to gene expression in CXCR4 virus. Expression of actin was used for normalization. Adjusted p-values were computed using the Bonferroni-Sidak method.

FIG. 3 shows that mice treated with CXCR4-miR virus exhibited a highly significant (p<0.01) reduction in expression of all tested viral genes in normal brain tissue, while retaining a high level of viral gene expression within the tumor. These results suggest that miRNA-dependent downregulation ICP34.5 gene expression reduces HSV-1 replication in normal brain tissue relative to brain tumor tissue.

### Example 2

### Expression of ICP34.5 in Neuronal and Tumor Cells

This Example show expression of the ICP34.5 protein in neuronal and in tumor cells following infection with either the CXCR4-miR virus or the control CXCR4 virus. Mouse neuronal cells, LNCap cells, and A549 cells were treated with either the CXCR4-miR virus or the CXCR4 virus. At 16 hrs post-infection, cells were pelleted, washed with Dulbecco's Phosphate Buffer Saline (PBS) and lysed by incubation in RIPA buffer (10 mM Tris-Cl (pH 8.0), 1 mM EDTA, 1% Triton X-100, 0.1% sodium deoxycholate, 0.1% SDS, 140 mM NaCl) with 1 mM phenylmethylsulfonyl fluoride (PMSF) and a protease inhibitor cocktail on ice for 40 minutes. Then, the lysate was centrifuged at 13,000 rpm for 10 minutes at 4′ C., and the supernatant was collected.

The level of ICP34.5 protein in each sample was determined by Western blot analysis. Total protein concentration was measured using the BSA assay. Protein lysates (30-40 μg) were mixed with 4×SDS loading dye, followed by heating at 95° C. for 10 minutes. Samples were then loaded and electrophoresed in 10% SDS-PAGE, followed by transfer to a nitrocellulose membrane. The membrane was subsequently blocked in Tris-buffered saline plus Tween 20 (TBST) with 5% BSA for 1 hour at room temperature. The blocked membrane was incubated overnight with an anti-ICP34.5 or β-actin antibody at 4° C. Then the membrane was washed with TBST for 3×10 minutes and incubated with a corresponding secondary antibody for 1 hour at room temperature. After three 10-minute washes using TBST, the membrane was incubated with enhanced chemiluminescence (ECL) reagents for 1 minute and then exposed in a BIO-RAD ChemiDoc XRS+ imaging system. Band intensities were quantified using ImageJ.

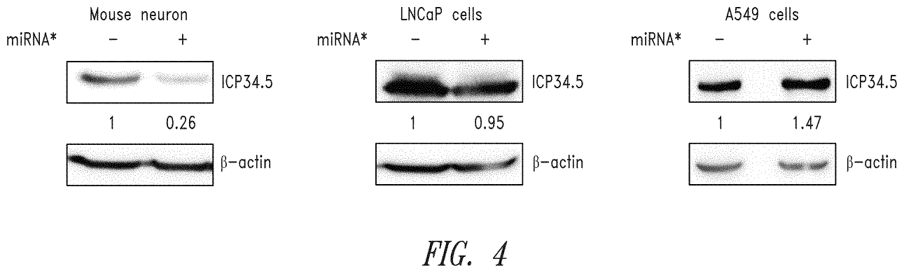

In FIG. 4, results of the Western blot are shown. The row labeled “miRNA” indicates whether cells were infected with a virus including (+) or lacking (−) miRNA binding elements in the 3′ UTR of the ICP34.5 gene. Expression of ICP34.5 was found to be lower in neuronal cells infected with a virus containing miRNA binding elements. In contrast, in tumor cells, expression was similar in cells infected with viral construct including or lacking the miRNA binding elements.

### Example 3

### MicroRNA-Based Oncolytic Virus Platform

This example presents a microRNA-based oncolytic virus platform with some exemplary engineered viral genomes. The platform is referred to herein as, “transcriptional and translational dual-regulation” (TTDR). The basic platform HSV-1-based vector is illustrated in FIG. 5. A key feature of the platform HSV-1 virus is translational control of the ICP34.5 gene by inserting five copies of the binding sites for miR-124 and miR-143 in the 3′-UTR of the ICP34.5 gene. Key elements of the platform virus vector may also include transcriptional control of the ICP27 gene, a gene essential for viral replication, using a tumor-specific promoter.

A wide variety of HSV-1 strains may be used as the backbone for construction of recombinant oncolytic viruses, including strain 17, strain KOS, strain F, strain McKrae, etc. All viral mutagenesis may be performed in Escherichia coli using standard lambda Red-mediated recombineering techniques implemented on the HSV-1 genome cloned into a bacterial artificial chromosome (BAC) (see generally: Tischer B K, Smith G A, Osterrieder N. Methods Mol Biol. 2010; 634:421-30. doi: 10.1007/978-1-60761-652-8_30. PMID: 20677001; Tischer B K, von Einem J, Kaufer B, and Osterrieder N., BioTechniques 40:191-197, February 2006 (including the Supplementary Material, doi: 10.2144/000112096; and Tischer B K, Smith, G A and Osterrieder N. Chapter 30, Jeff Braman (ed.), In Vitro Mutagenesis Protocols: Third Edition, Methods in Molecular Biology, vol. 634, doi: 10.1007/978-1-60761-652-8_30, Springer Sceince+Business Media, LLC 2010).

Tumor-specific promoters may also be used to drive expression of a cassette encoding the immunomodulators IL12/IL15/IL15RA, which boost the anti-tumor immune response. The immunomodulator expression cassette may be controlled by a hCEA, hCXCR4, or PSA promoter and be inserted into the viral genome in a location which does not have a negative impact on viral gene expression and replication, such as between viral genes US1/US2, UL3/UL4 and /or UL50/UL51. To facilitate in vivo testing in a variety of mouse models, other recombinant viruses may be constructed expressing murine IL12 instead of human IL12. Human IL15 can be retained in mouse-specific oncolytic viruses due to its activity in mouse cells.

The vectors may include an expression cassette encoding a fusogenic form of the Gibbon ape leukemia virus (GALV) env protein lacking the C-terminal R-peptide, which enhances virus cytotoxicity. Alternatively, the expression cassette can encode a fusogenic form of glycoprotein B (e.g., truncated gB 876t). The cassette may be inserted into the viral genome in a location which does not have a negative impact on viral gene expression and replication such as between viral genes US1/US2, UL3/UL4 and /or UL50/UL51.

The viral vectors also may include an expression cassette for the HSV-1 thymidine kinase (TK) gene inserted between viral genes US1/US2, UL3/UL4 and /or UL50/UL51. If a BAC sequence is inserted into the viral genome to facilitate mutagenesis in E. coli, disrupting the native TK gene. Presence of the exogenous TK gene enhances virus safety by rendering the virus sensitive to common treatment with guanosine analogues, such as ganciclovir and acyclovir.

The promoter driving expression of the key HSV-1 transcriptional regulator ICP27 may be replaced with a tumor-specific promoter such as hCEA, hCXCR4, PSA, or Probasin (ARR2PB). The 3′ UTR of the viral gene encoding the neurovirulence factor ICP34.5 may also be modified by insertion of multiple copies of microRNA recognition elements to abrogate production of ICP34.5 in tissues containing high levels of the corresponding microRNA. In an exemplary embodiment, five copies of miR-124 and five copies of miR-143 recognition elements may be inserted in tandem into the 3′ UTR of ICP34.5.

The terminal repeat region of the viral genome may be completely deleted to reduce the overall genome size and create more space for transgene insertions; the deleted TR is engineered to avoid disrupting the native promoter of the ICP47 gene, which is normally part of the terminal repeats. Further details of exemplary elements discussed herein are illustrated in FIG. 6.

The resulting recombinant viruses may be isolated using the Qiagen HiSpeed MidiPrep Kit and transfected into Vero cells to recover the virus, e.g. using Lipofectamine 2000. Targeted sequencing of all modified regions and restriction profiling may be used to verify genomic integrity. Stability of the final recombinant viruses may be confirmed by serial passaging and periodic verification of transgene expression by Western blot and ELISA.

Five exemplary embodiments for this platform are set forth in the table below. Two viruses are engineered to be used for the treatment of lung cancer (or other cancers of epithelial cell origin, e.g., renal, and breast cancer) and three for the treatment of prostate cancer.

| | | | | | Optional | |
|---|---|---|---|---|---|---|
| Tumor | Virus | ICP27 | IL12/IL15/IL15RA | miR in 3′UTR | GALV | TR |
| Target | Name | promoter | promoter | of ICP34.5 | inserted | deleted |
| Lung | hVG182LF | hCEA | hCXCR4 | 5x miR-124 & | YES | YES |
| | | | | 5xmiR-143 | | |
| Lung | hVG185LF | hCXCR4 | hCEA | 5x miR-124 & | YES | YES |
| | | | | 5xmiR-143 | | |
| Prostate | hVG183PF | hCXCR4 | PSA | 5x miR-124 & | YES | YES |
| | | | | 5xmiR-143 | | |
| Prostate | hVG184PF | PSA | hCXCR4 | 5x miR-124 & | YES | YES |
| | | | | 5xmiR-143 | | |
| Prostate | hVG189PF | Probasin | hCXCR4 | 5x miR-124 & | YES | YES |
| | | (ARR2PB) | | 5xmiR-143 | | |

### Example 4

### MicroRNA-Mediated Regulation of ICP34.5 Expression Leads to Reduced Neurovirulence In Vivo

Mice were injected intracranially with a single dose (5×10̂7 PFU/mL) of either a CXCR4-TF-Fc-h1215-miR virus, in which five miR-124 and miR-143 elements are inserted into the 3′ UTR of the ICP34.5a gene, or a control CXCR4-TF-Fc-h1215 virus lacking this insertion. Both viral constructs also include a CXCR4 promoter-driven ICP27 gene, a TF+Fc PD-L1 blocker expression cassette inserted between UL3 and UL4, and a terminal repeat region replaced with a cassette expressing human IL12, IL15, and IL15 receptor alpha subunit.

Following infection, the extent of HSV-1 infection was visualized by staining murine brain sections with rabbit polyclonal anti-HSV primary antibody and rat anti-rabbit secondary antibody conjugated to AlexaFluor 488. As shown in FIG. 7, mice infected with virus containing miR-controlled ICP34.5 showed detectable virus only along the needle path, while the virus containing wild-type ICP34.5 was widely disseminated throughout the brain.

### Example 5

### The VG182LF Virus Selectively Kills Lung Cancer Cells In Vitro

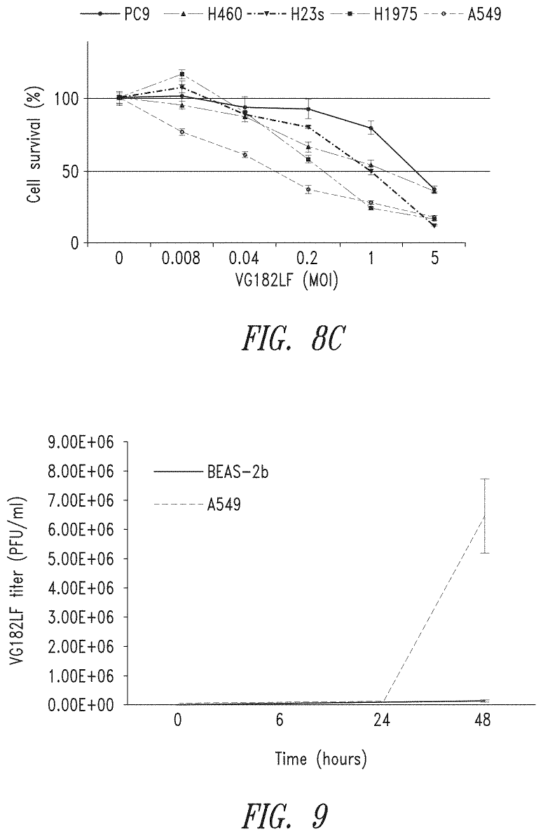

Lung cancer cells (A549) or normal lung cells (BEAS-2b and HPL1D) were incubated with VG182LF virus at increasing MOIs for 72 hours. Following infection, cell viability was measured using the MTT assay. As shown in FIGS. 8A and 8B, VG182LF virus demonstrates increased killing of lung cancer cells relative to normal lung cells in a dose-dependent manner.

The table below presents the IC50 values determined for each cell line and shows that there is a 6.54-fold and 18.93-fold increase in IC50 for the normal lung cells HPL1D and BEAS-2b, respectively, as compared to the lung cancer cell line, A549.

| | IC50 value | IC50 fold-increase compared |
|---|---|---|
| Cell lines | (MOI) | to A549 cells |
| A549 | 0.0974 | |
| BEAS-2b | 1.844 | 18.93 |
| HPL1D | 0.637 | 6.54 |

These data indicate that increased tumor cell killing is linked to microRNA control of ICP34.5 gene expression and to the use of tumor-specific promoters to drive expression of the ICP27 and of IL12/IL15/IL15RA genes.

This experiment was repeated with additional lung cancer cell lines. As shown in FIG. 8C, the VG182LF virus efficiently kills a wide variety of commercially available lung cancer cells. Calculated IC50 values for each cell line are set forth in the table below.

| | IC50 (MOI) | |
|---|---|---|
| Cell line | VG182LF | VG182LGsnp |
| A549 | 0.0975 | 0.0646 |
| H23S | 0.876 | 0.6135 |
| H1975 | 0.32705 | 0.0856 |
| PC9 | 3.165 | 2.8975 |
| H460 | 1.306 | 0.8055 |

### Example 6

### VG182LF Selectively Replicates in Lung Cancer Cells In Vitro

Lung cancer cells (A549) and normal lung cells (BEAS-2b) were treated with the VG182LF virus at a MOI of 0.1 for different times. Following infection, viruses were harvested and titrated on Vero cells. As shown in FIG. 9, the VG182LF virus successfully replicates in lung cancer cells, but not in normal lung cells. At the 48 hour time point, a titer of greater than 6×10⁶ viral particles was obtained from the A549 lung cancer cells; while no significant virus was obtained from the BEAS-2b normal lung cells, suggesting that microRNA control of ICP34.5 and the use of tumor-specific promoters to drive expression of ICP27 and of IL12/IL15/IL15RA negatively impacts viral replication in normal cells, while promoting viral replication in tumor cells.

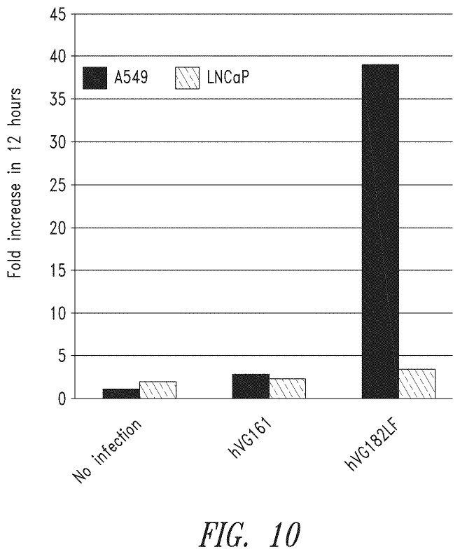

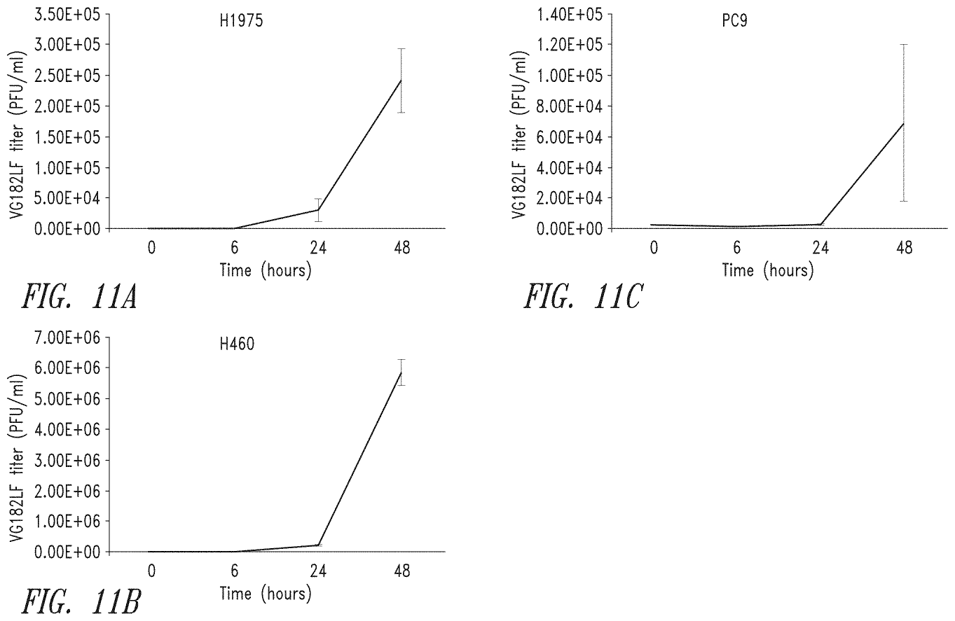

Replication of the VG182LF virus in A549 lung tumor cells or LNCaP prostate tumor cells was studied. Briefly, cells were infected with either the VG161 (control) or VG182LF virus for 12 or 24 hours. Cells were subsequently harvested and intracellularly stained with anti-human IL-12p70 antibody. Human IL-12 positive cells were detected by flow cytometry, and the fold increase of human IL-12 was calculated. As shown in FIG. 10, increased expression of human IL-12 directly correlates to with enhanced virus replication.

The ability of the VG182LF virus to replicate in a variety of different lung cancer cells lines was assessed. Cells from lung cancer cell lines H1975, PC9 and H460 were treated with the VG182LF virus at an MOI of 0.1, and supernatant was harvested at 0, 6, 24, and 48 hours post-infection. The virus from each sample was titrated on Vero cells. The data from this experiment are shown in FIGS. 11A-C with titer values representing the average of 3 biological replicates. These data indicate that at 48 following infection, the virus was able to replicate to a significant level in each lung cancer cell line.

### Example 7

### VG182LF In Vivo Anti-Tumor Efficacy in H1975 Lung Cancer Model

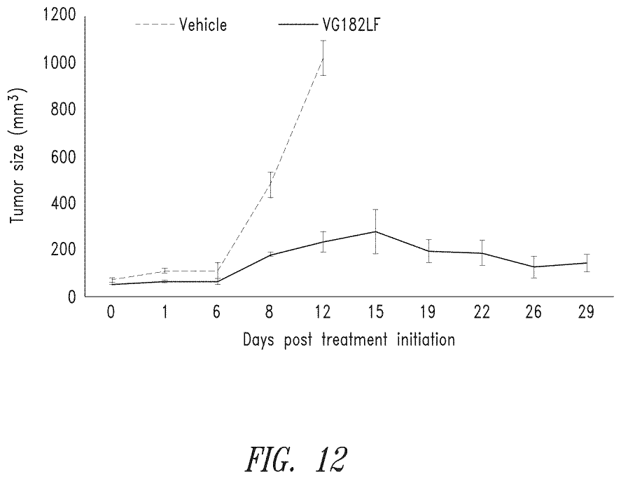

Nude mice bearing H1975 tumors were treated with VG182LF one week post-implantation. 5.65×10̂7 PFU/mouse of VG182LF was injected 3 times at 2-day intervals. Vehicle-treated mice reached a humane endpoint and were sacrificed 12 days after treatment initiation. As shown in FIG. 12, mice treated with the VG182LF virus showed dramatically reduced tumor growth compared to vehicle-treated controls and were still alive by 29 days post treatment initiation.

### Example 8

### miR-Mediated Control of ICP34.5 Expression in Transfected Cells in Culture

The HSV-1 protein ICP34.5 is required for effective viral replication in neurons, but is largely dispensable for replication in non-neuronal cells in culture, such as 293FT cells. In this Example, the ability of miR-143 to influence the expression of ICP34.5 in 293FT cells was assessed.

Cells were initially transfected with miR-143 on day 0. As controls, 293FT cells were either transfected with scrambled miR or not transfected at all. 20 hours post-transfection, cells were washed and then infected with a recombinant oncolytic HSV-1 (MOI=1), which encodes binding sites for miR-143 and miR-124 in the 3′ UTR of ICP34.5 along with a fusogenic mutation in the carboxyl terminus of gB (gB-876t). Cells were harvested at 6 hours post-infection for RNA isolation to measure gene expression and transfection efficiency and at both 0 hours and 24 hours post-infection for DNA isolation to measure virus replication.

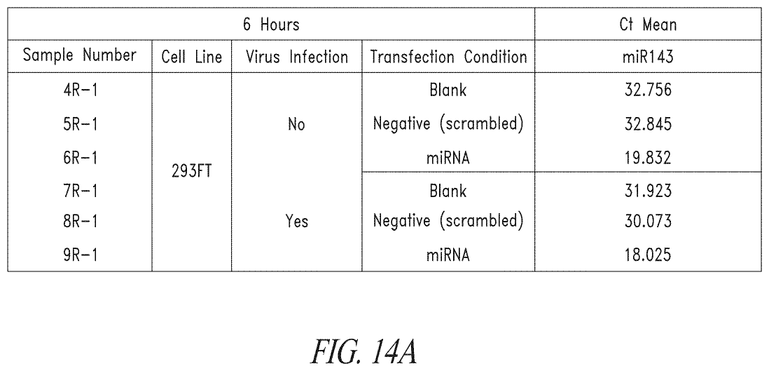

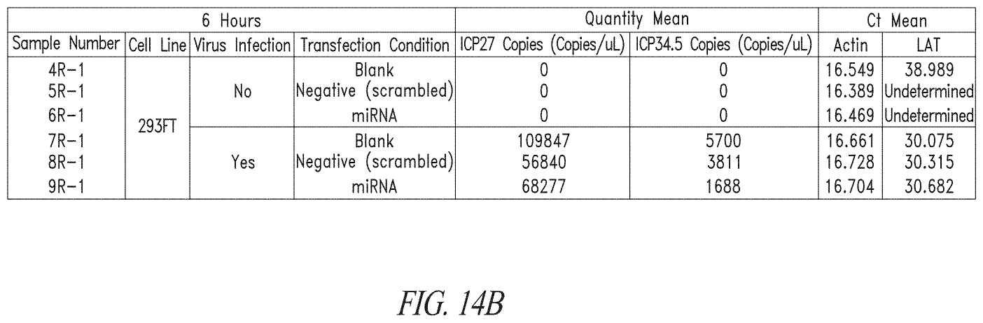

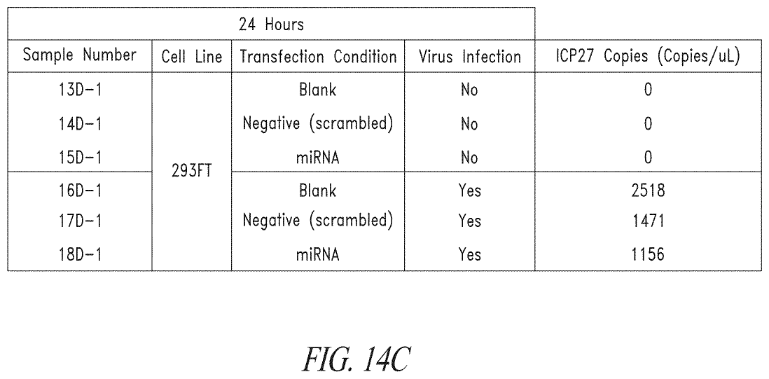

As shown in FIG. 14A, high levels of miR-143 were detected at 6 hours post-infection by RT-qPCR in cells transfected with miR-143, while non-transfected cells and cells transfected with scrambled miR showed negligible levels of miR-143. As shown in FIG. 14B, viral gene expression evaluated at 6 hours post-infection by RT-qPCR revealed a significant decrease in ICP34.5 expression in samples that were previously transfected with miR-143, while a similar decrease was not observed with another viral gene (ICP27) that does not contain miR binding sites. Virus replication was quantified at 24 hours post-infection by using qPCR to measure copies of ICP27, with each copy corresponding to a discrete viral genome. As shown in FIG. 14C, levels of viral replication were not significantly different when comparing samples transfected with miR-143 or with scrambled miR, suggesting that the dramatic reduction observed in expression of ICP34.5 in samples transfected with miR-143 was not due to reduced virus copy number.

### Example 9

### miR Regulation of ICP34.5 Improves Safety by Blocking Neurovirulence

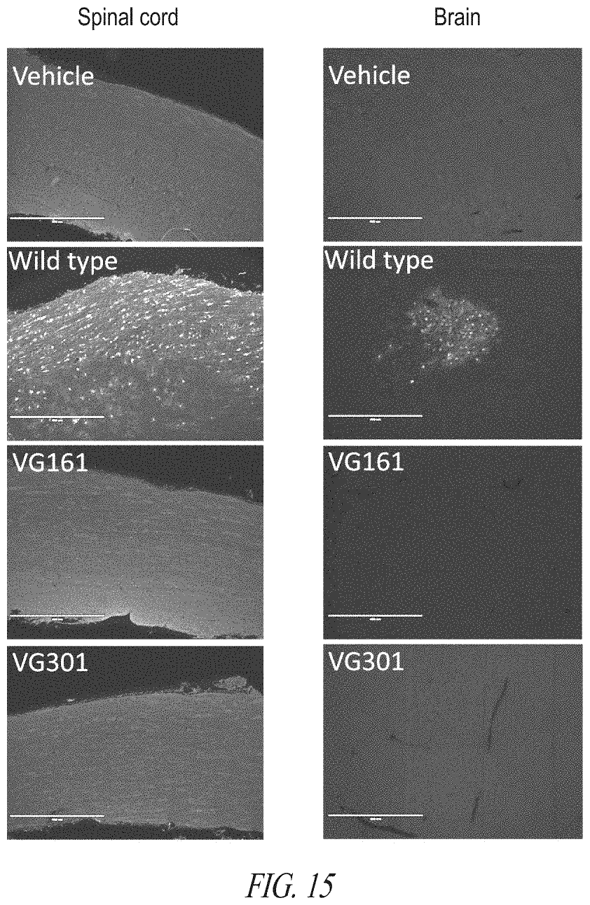

In this Example, DBA/2 mice (N=3 for each group) were injected subcutaneously with either a vehicle control, wild-type HSV-1, the VG161 virus variant with deleted ICP34.5 and no fusion mutation, or with the VG301 virus variant which encodes binding sites for miR-143 and miR-124 in the 3′ UTR of ICP34.5 along with a fusogenic mutation in the carboxyl terminus of gB (gB-876t). Samples were harvested at 6 days post-injection for HSV-1 immunostaining. As shown in FIG. 15, a pattern of robust viral replication was observed in both the brain and spinal cord of mice injected with wild-type HSV-1. In contrast, no viral replication was observed in neuronal tissues of mice injected with either the VG161 or VG301 variants, suggesting that miR regulation of ICP34.5 is just as effective as complete deletion of ICP34.5 in preventing neurovirulence.

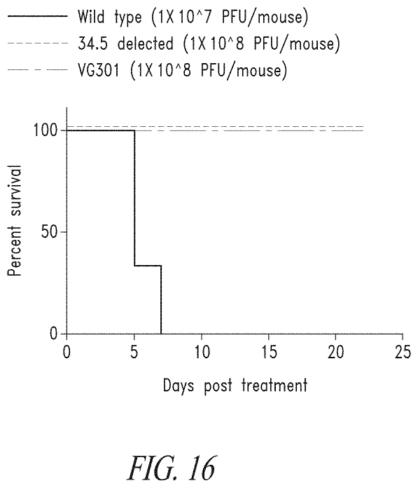

The remaining mice that were injected with wild-type HSV-1 rapidly developed neurological symptoms and had to be euthanized, while all remaining mice that were treated with either the VG161 or VG301 variants remained healthy for the full duration of the experiment, as shown in FIG. 16. These results provide additional evidence supporting the safety and efficacy of using miR regulation of ICP34.5 to prevent OV-induced neurovirulence. Furthermore, it can be concluded that the fusogenic mutation in VG301 does not lead to increased morbidity or mortality.

### Example 10

### Assessment of a Fusogenic Mutation in Oncolytic HSV-1

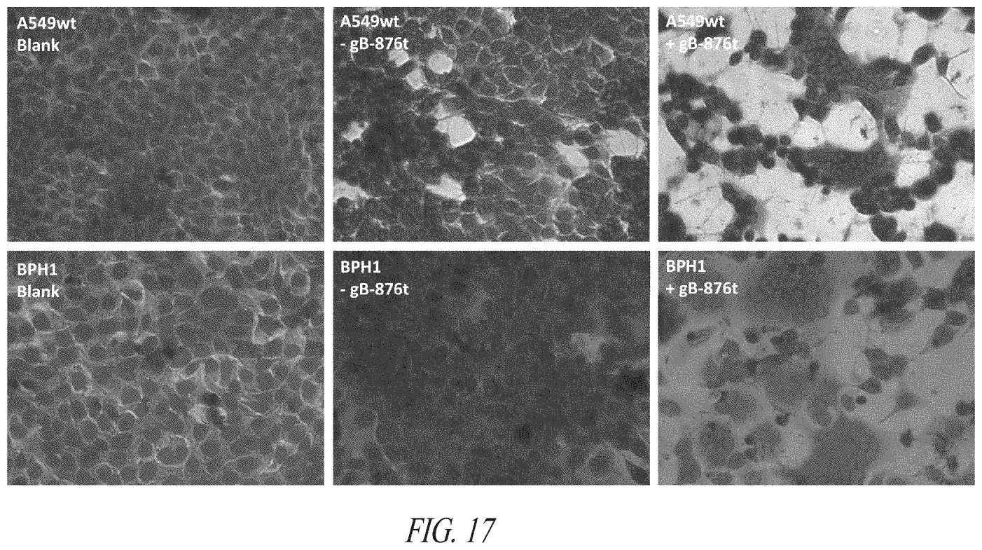

In this Example, A549wt and BPH1 cells were infected with a recombinant oncolytic HSV-1, which encodes a fusogenic mutation in the carboxyl terminus of gB (+gB-876t). As a control, A549wt and BPH1 cells were infected with HSV-1 lacking the fusogenic mutation (−gB-876t). At 48 hours post-infection, cells were fixed and Giemsa stained to visualize viral plaques and syncytia resulting from virus-induced cell fusion. As shown in FIG. 17, massive amounts of cell-to-cell fusion were observed in cells infected with the virus carrying the fusogenic mutation, while minimal fusion was evident in cells infected with virus lacking the fusogenic mutation.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. A recombinant herpes simplex virus comprising a modified oncolytic herpes virus genome, wherein the modified herpes virus genome comprises at least one miRNA target sequence operably linked to a first, or, to a first and a second copy of an ICP34.5 gene.

2. The recombinant herpes simplex virus according to claim 1 wherein the miRNAs are selected from the group consisting of mIR-122, miR-124, miR-124*, miR-127, miR-128, miR-129, miR-129*, miR-132, mIR-133a, mIR133b, miR-135b, miR-136, miR-136*, miR-137, miR-139-5p, miR-143, mIR-145, miR-154, miR-184, miR-188, miR-204, mIR216a, miR-299, miR-300-3p, miR-300-5p, miR-323, miR-329, miR-337, miR-335, miR-341, miR-369-3p, miR-369-5p, miR-376a, miR-376a*, miR-376b-3p, miR-376b-5p, miR-376c, miR-377, miR-379, miR-379*, miR-382, miR-382*, miR-409-5p, miR-410, miR-411, miR-431, miR-433, miR-434, miR-451, miR-466b, miR-485, miR-495, miR-539, miR-541, miR-543*, miR-551b, miR-758, and miR-873.

3. The recombinant herpes simplex virus of claim 2 wherein the miRNA target sites comprise five copies of the binding sites for miR-124 and miR-143.

4. The recombinant herpes simplex virus of claim 1, wherein the oncolytic herpes virus is HSV-1.

5. The recombinant herpes simplex virus of claim 4, wherein the genome further comprises a fusogenic mutation in a gene encoding glycoprotein B (gB).

6. The recombinant herpes simplex virus of claim 5, wherein the gene encoding for glycoprotein B (gB) encodes a glycoproptein B variant that terminates after amino acid 876.

7. The recombinant herpes simplex virus of claim 1, wherein the modified oncolytic herpes virus genome comprises additional mutations or modifications in at least one viral gene selected from the group consisting of ICP6, ICPO, ICP4, ICP27, ICP47, ICP24, and ICP56.

8. The recombinant herpes simplex virus of claim 7, wherein said at least one viral gene is modified by replacement of the native promoter.

9. The recombinant herpes simplex virus of claim 1, further comprising at least one nucleic acid encoding a non-viral protein selected from the group consisting of immunostimulatory factors and checkpoint blocking peptides, wherein the at least one nucleic acid is operably linked to a tumor-specific promoter.

10. The recombinant herpes simplex virus of claim 9, wherein the non-viral protein is selected from the group consisting of IL12, IL15, IL15 receptor alpha subunit, OX40L, and a PD-L1 blocker.

11. A method for lysing tumor cells, comprising providing a therapeutically effective amount of recombinant herpes simplex virus according to claim 1.

12. A therapeutic composition comprising the recombinant herpes simplex virus according to claim 1 and a pharmaceutically acceptable carrier.

13. A method for treating cancer in a subject suffering therefrom, comprising the step of administering a therapeutically effective amount of the composition of claim 12.
